(19) United States
(12) Patent Application Publication
(10) Pub. No.: US 2014/0271518 A1
(43) Pub. Date: Sep. 18, 2014

(54) COSMETIC AEROSOL SPRAY WITH LASTING FRESHNESS EFFECT

(71) Applicant: Henkel AG & Co. KGaA, Duesseldorf (DE)

(72) Inventors: Bernhard Banowski, Duesseldorf (DE); Imme Breuer, Duesseldorf (DE)

(73) Assignee: Henkel AG & Co. KGaA, Duesseldorf (DE)

(21) Appl. No.: 14/291,290

(22) Filed: May 30, 2014

Related U.S. Application Data
(63) Continuation of application No. PCT/EP2012/073031, filed on Nov. 20, 2012.

(30) Foreign Application Priority Data
Dec. 2, 2011 (DE) 10 2011 087 662.6

(51) Int. Cl. A61K 8/26; A61K 8/06; A61Q 15/00; A61K 8/42

(52) U.S. Cl. CPC A61K 8/26; A61K 8/42; A61K 8/064; A61Q 15/00
USPC 424/68

(57) ABSTRACT

A water-in-oil emulsion including a) at least one sweat-inhibiting aluminum salt, b) at least one water-in-oil emulsifier having an HLB value in the range from 1.5 to 6.5, c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, d) at least one cosmetic oil that is not a fragrance and not an essential oil, e) 5-methyl-2-(1-methylethyl)cyclohexyl-N-ethyl oxamate, preferably (1R,2S,5R)-5-methyl-2-(1-methylethyl)cyclohexyl-N-ethyl oxamate, at least one propellant, and an aerosol dispensing device have high emulsion stability due to a low corrosive tendency, a good cosmetic effect, a lasting freshness effect on the skin, good skin compatibility and low residue formation during and after application.

### FIELD OF THE INVENTION

The present invention generally relates to aerosol-sprayable cosmetic water-in-oil emulsions having a lasting freshness effect.

### BACKGROUND OF THE INVENTION

An aerosol is a disperse system in which a solid or a liquid is very finely divided in a gas. The aerosol itself is generally only produced on application with the aid of a suitable spray system, by spraying solutions, emulsions or suspensions, to which end spray cans for example can be used in which a liquefied pressurized gas serves as a propellant gas. When the pressure valve is opened, the propellant-preparation mixture is released through a fine nozzle and the propellant evaporates, leaving behind the finely divided spray product as an aerosol.

A technically important field for the use of cosmetic aerosol sprays is the area of deodorizing cosmetic products. Common deodorant spray compositions frequently take the form of anhydrous ethanolic solutions of the deodorizing active ingredient. One disadvantage of these ethanolic solutions is a skin-irritating effect in correspondingly predisposed persons with sensitive skin. The application of common ethanolic solutions likewise leads to a very unpleasant burning on skin that has been mechanically irritated by shaving. A further disadvantage of ethanolic solutions is that there is little or no possibility of incorporating water-containing or water-soluble active ingredients, in particular antiperspirant or deodorant active ingredients or hair growth-inhibiting active ingredients, that are not also soluble in ethanol.

As an alternative presentation form to ethanolic solutions, anhydrous suspensions of the powdered sweat-reducing active ingredient, mostly an aluminum salt, in addition to the propellant gas in a liquid carrier, mostly a relatively volatile oil such as cyclomethicone, are discussed in the prior art. The suspension has to be shaken before being sprayed. One disadvantage of these suspension aerosols is the risk that the valve or nozzle holes become blocked if the salt is used in relatively high concentrations. Attempts have therefore been made to spray the antiperspirant salt in dissolved form. However, the packaging of aqueous antiperspirant salt solutions in propellant-containing metal cans gives rise to severe corrosion problems in the aerosol packaging, such that corrosion phenomena inevitably occur even on lacquered spray cans.

The prior art describes water-in-oil emulsions (W/O emulsions) as a further alternative presentation form. Thus the German patent application DE 102006062499 A1 (Henkel) discloses a W/O emulsion that is suitable as an aerosol spray.

Sweat-inhibiting W/O emulsion sprays have particularly skin-caring properties. When sprayed onto the skin the propellant gas temporarily creates a slightly cooling effect, although this does not last. The cooling effect created by the aqueous phase of the emulsion included in the spray is scarcely perceptible in a W/O emulsion in which the aqueous phase is the internal phase. In addition to highly volatile substances that produce an evaporative coldness, substances that stimulate the heat receptors in the skin and mucous membranes such that a cool sensory impression is formed can also give rise to a skin-cooling effect. In particular, the CMR-1 receptor (cold- and menthol-sensitive receptor), which belongs to the family of TRP channels, is stimulated by such cooling active ingredients, producing an impression of coldness. The best-known such cooling active ingredient is undoubtedly L-menthol. Although L-menthol is a highly efficient cooling active ingredient, it has a number of disadvantages: the cooling effect is not very lasting and moreover the substance has a marked inherent odor, which counteracts a variable perfuming of the cosmetic products with as wide a variety as possible of scents. Furthermore, L-menthol can provoke undesired skin reactions in sensitive persons.

It is therefore desirable to provide a stable water-in-oil emulsion which is suitable for use in an aerosol spray and has a content of sweat-inhibiting aluminum salts and which brings about a long-lasting cooling and freshness effect on the skin and avoids the disadvantages of the cooling active ingredient L-menthol as described above.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cosmetic product consisting of: a water-in-oil emulsion including a) at least one sweat-inhibiting aluminum salt, b) at least one water-in-oil emulsifier having an HLB value in the range from 1.5 to 6.5, c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, d) at least one cosmetic oil that is not a fragrance and not an essential oil, e) 5-methyl-2-(1-methylethyl)cyclohexyl-N-ethyl oxamate, preferably (1R,2S,5R)-5-methyl-2-(1-methylethyl)cyclohexyl-N-ethyl oxamate; at least one propellant; and an aerosol dispensing device.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It was established that the object described can be achieved by means of sweat-inhibiting water-in-oil emulsion sprays including 5-methyl-2-(1-methylethyl)cyclohexyl-N-ethyl oxamate as a cooling active ingredient.

The present application therefore provides a cosmetic product consisting of
a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17,
d) at least one cosmetic oil that is not a fragrance and not an essential oil,
e) 5-methyl-2-(1-methylethyl)cyclohexyl-N-ethyl oxamate, preferably (1R,2 S,5R)-5-methyl-2-(1-methylethyl) cyclohexyl-N-ethyl oxamate,
at least one propellant,
an aerosol dispensing device.

Unless otherwise specified, all stated amounts in this application relate to the weight of the propellant-free water-in-oil emulsion according to the invention.

The water-in-oil emulsions according to the invention include at least one sweat-inhibiting aluminum salt.

Preferred sweat-inhibiting aluminum salts are selected from the water-soluble astringent inorganic and organic salts of aluminum and any mixtures of these salts. Alumosilicates and zeolites are not included in the sweat-inhibiting aluminum salts according to the invention. According to the invention water solubility is understood to mean a solubility of at least 3 wt. % at 20° C., in other words amounts of at least 3 g of the antiperspirant active ingredient are soluble in 97 g of water at 20° C.

Particularly preferred sweat-inhibiting aluminum salts are selected from aluminum chlorohydrate, in particular aluminum chlorohydrate of the general formula [Al₂(OH)5Cl·1-6 H2O]ₙ, preferably [Al₂(OH)₅Cl·2-3 H2O]ₙ, which can be present in non-activated or in activated (=depolymerized) form, and aluminum chlorohydrate of the general formula [Al2(OH)₄Cl₂·1-6 H₂O]ₙ, preferably [Al₂(OH)₄Cl₂·2-3 H₂O]ₙ, which can be present in non-activated or in activated (=depolymerized) form.

Also preferred are aluminum sesquichlorohydrate, aluminum dichlorohydrate or aluminum chlorohydrex polyethylene glycol (PEG), aluminum glycol complexes, e.g. aluminum propylene glycol complexes, aluminum sesquichlorohydrex PG, aluminum sesquichlorohydrex PEG or aluminum PEG dichlorohydrex, aluminum hydroxide, selected moreover from potassium aluminum sulfate (KAI(SO4)₂•12 H2O, alum), aluminum undecylenoyl collagen amino acid, sodium aluminum lactate+aluminum sulfate, sodium aluminum chlorohydroxylactate, aluminum bromohydrate, aluminum chloride, the aluminum salts of lipoamino acids, aluminum sulfate, aluminum lactate, aluminum chlorohydroxyallantoinate and sodium aluminum chlorohydroxylactate.

Sweat-inhibiting aluminum salts that are particularly preferred according to the invention are selected from “activated” aluminum salts, which are also referred to as antiperspirant active ingredients with enhanced activity. Activated aluminum salts are generally produced by heat treating a relatively dilute solution of the salt (e.g. approximately 10 wt. % salt) to increase its HPLC peak 4 to peak 3 surface area ratio. The activated salt can then be dried, in particular spray-dried, to form a powder. In addition to spray drying, drum drying for example is also suitable. Activated aluminum salts typically have an HPLC peak 4 to peak 3 surface area ratio of at least 0.4, preferably at least 0.7, more preferably at least 0.9, wherein at least 70% of the aluminum can be assigned to these peaks.

Preferred amino acids for stabilizing the sweat-inhibiting salts are selected from glycine, alanine, leucine, isoleucine, β-alanine, valine, cysteine, serine, tryptophan, phenylalanine, methionine, β-amino-n-butanoic acid and γ-amino-n-butanoic acid and the salts thereof, in each case in the d form, 1 form or d1 form; glycine is particularly preferred.

Preferred hydroxyalkanoic acids for stabilizing the sweat-inhibiting salts are selected from glycolic acid and lactic acid.

Further preferred sweat-inhibiting active ingredients are activated aluminum salts including 5 to 78 wt. % (USP) of an activated sweat-inhibiting aluminum salt, an amino acid or hydroxyalkanoic acid in an amount to provide a weight ratio of (amino acid or hydroxyalkanoic acid) to Al of 2:1 to 1:20 and preferably 1:1 to 1:10, and a water-soluble strontium salt in an amount to provide a weight ratio of Sr to Al of 1:1 to 1:28 and preferably 1:2 to 1:25.

Particularly preferred solid sweat-inhibiting activated salt compositions include, relative to their weight, 48 to 78 wt. % (USP), preferably 66 to 75 wt. % of an activated aluminum salt and 1 to 16 wt. %, preferably 4 to 13 wt. % of molecularly bound water, also sufficient water-soluble strontium salt that the weight ratio of Sr to Al is 1:1 to 1:28, preferably 1:2 to 1:25, and sufficient amino acid that the weight ratio of amino acid to Al is 2:1 to 1:20, preferably 1:1 to 1:10.

Further particularly preferred solid sweat-inhibiting activated salt compositions include, relative to their weight, 48 to 78 wt. % (USP), preferably 66 to 75 wt. % of an activated aluminum salt and 1 to 16 wt. %, preferably 4 to 13 wt. % of molecularly bound water, also sufficient water-soluble strontium salt that the weight ratio of Sr to Al is 1:1 to 1:28, preferably 1:2 to 1:25, and sufficient glycine that the weight ratio of glycine to Al is 2:1 to 1:20, preferably 1:1 to 1:10.

Further particularly preferred solid sweat-inhibiting activated salt compositions include, relative to their weight, 48 to 78 wt. % (USP), preferably 66 to 75 wt. % of an activated aluminum salt and 1 to 16 wt. %, preferably 4 to 13 wt. % of molecularly bound water, also sufficient water-soluble strontium salt that the weight ratio of Sr to Al is 1:1 to 1:28, preferably 1:2 to 1:25, and sufficient hydroxyalkanoic acid that the weight ratio of hydroxyalkanoic acid to Al is 2:1 to 1:20, preferably 1:1 to 1:10.

Further preferred activated aluminum salts are those of the general formula Al₂(OH)₆₋ₐXa, in which X is Cl, Br, 1 or NO₃ and “a” is a value from 0.3 to 5, preferably from 0.8 to 2.5 and particularly preferably 1 to 2, such that the molar ratio of Al to X is 0.9:1 to 2.1:1. These salts generally include a little associatively bound water of hydration, typically 1 to 6 mol of water per mol of salt. Aluminum chlorohydrate is particularly preferred (i.e. X is Cl in the above formula), and specifically 5/6-basic aluminum chlorohydrate, wherein “a” is 1, such that the molar ratio of aluminum to chlorine is 1.9:1 to 2.1:1.

Particularly preferred water-in-oil emulsions according to the invention include the at least one antiperspirant active ingredient in a total amount from 5 to 40 wt. %, preferably 10 to 30 wt. %, particularly preferably 15 to 25 wt. % and exceptionally preferably 20 to 23 wt. %, relative in each case to the total weight of the active substance (USP), free from water of crystallization and ligands, in the propellant-free water-in-oil emulsion.

In a particularly preferred embodiment the water-in-oil emulsion includes an astringent aluminum salt, in particular aluminum chlorohydrate, particularly preferably aluminum chlorohydrate having an active substance (USP) free from water of crystallization of 72 to 88 wt. %, relative to the raw material as is. In a further particularly preferred embodiment the water-in-oil emulsion includes a non-activated astringent aluminum salt, in particular aluminum chlorohydrate, particularly preferably aluminum chlorohydrate having an active substance (USP) free from water of crystallization of 72 to 88 wt. %, relative to the raw material as is. Preferred non-activated aluminum chlorohydrates are for example Chlorhydrol® (Summit-Reheis), ACH 303 (Summit-Reheis), Locron® L (Clariant) or Aloxicoll® L (Giulini). A preferred activated aluminum chlorohydrate solution is for example Reach® 501 from Summit-Reheis.

The present application preferably provides a cosmetic product consisting of
a water-in-oil emulsion including
a) at least one sweat-inhibiting aluminum salt in a total amount from 5 to 40 wt. %, preferably 10 to 30 wt. %, particularly preferably 15 to 25 wt. % and exceptionally preferably 20 to 23 wt. %, relative in each case to the total weight of the active substance (USP), free from water of crystallization and ligands, in the propellant-free water-in-oil emulsion,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17,
d) at least one cosmetic oil that is not a fragrance and not an essential oil,
e) 5-methyl-2-(1-methylethyl)cyclohexyl-N-ethyl oxamate, preferably (1R,2S,5R)-5-methyl2-(1-methylethyl)cyclohexyl-N-ethyl oxamate,
at least one propellant,
an aerosol dispensing device.

The water-in-oil emulsions according to the invention include as the second essential constituent at least one water-in-oil emulsifier. Within the meaning of the present application an interfacially active substance having an HLB value under normal conditions in the range from 1.5 to 6.5, preferably 4 to 6.5, is regarded as a water-in-oil emulsifier. “Normal conditions” within the meaning of the present application are a temperature of 20° C. and a pressure of 1013.25 hPa. Melting point details likewise relate to a pressure of 1013.25 hPa.

Water-in-oil emulsions in which the at least one water-in-oil emulsifier b) is included in a total amount from 0.2 to 4.0 wt. %, preferably 0.4 to 3.0 wt. % and in particular 0.7 to 2.0 wt. %, relative in each case to the weight of the total propellant-free water-in-oil emulsion, are preferred according to the invention.

Water-in-oil emulsifiers that are preferred according to the invention are poly(C₂-C₃)alkylene glycol-modified silicones that are hydrophobically modified with C₄-C₁₈ alkyl groups, particularly preferably cetyl PEG/PPG-10/1 dimethicone (formerly INCI name Cetyl Dimethicone Copolyol), obtainable for example as Abil EM 90 or in a mixture of polyglyceryl-4-isostearate, cetyl PEG/PPG-10/1 dimethicone and hexyl laurate under the trade name Abil WE 09 from Evonilc, and moreover preferably lauryl PEG/PPG-18/18 methicone, obtainable for example as Dow Corning 5200 Formulation Aid.

A group of water-in-oil emulsifiers that are particularly preferred according to the invention are poly-(C₂-C₃)alkylene glycol-modified silicones whose former INCI name was dimethicone copolyol, preferably poly-(C₂-C₃)alkylene glycol-modified silicones having the INCI names PEG-x dimethicone with x=2-20, preferably 3-17, particularly preferably 11-12, bis-PEG-y dimethicone with y=3-25, preferably 4-20, PEG/PPG-a/b dimethicone, where a and b independently of one another denote numbers from 2-30, preferably 3-24, particularly preferably 12-20, in particular 14-18, bis-PEG/PPG-c/d dimethicone, where c and d independently of one another denote numbers from 10-25, preferably 14-20 and particularly preferably 14-16, and bis-PEG/PPG-e/f PEG/PPG-g/h dimethicone, where e, f, g and h independently of one another denote numbers from 10-20, preferably 14-18 and particularly preferably 16, on condition that these ethoxylated and/or propoxylated silicone emulsifiers have an HLB value under normal conditions in the range from 1.5 to 6.5, preferably 4 to 6.5.

For clarification it should be noted that these aforementioned preferred water-in-oil emulsifiers b) are alkylated only with methyl groups and—as is already implied by the names “dimethicone copolyol” and “PEG/PPG dimethicone”—include no higher alkyl groups, for example ethyl, lauryl, cetyl, in the molecule.

A water-in-oil emulsifier that is exceptionally preferred according to the invention is PEG/PPG-18/18 dimethicone, which has an HLB value in the range from 4 to 6.5. This particularly preferred water-in-oil emulsifier PEG/PPG-18/18 dimethicone is not usually available commercially as a pure substance but rather dispersed in a cosmetic oil. Preferred products according to the invention comprise a water-in-oil emulsion including the water-in-oil emulsifier PEG/PPG-18/18 dimethicone as a dispersion in dimethicone having a viscosity from 3 to 100 cSt, preferably 5 to 50 cSt, particularly preferably 10 to 20 cSt (at 25° C. in each case). A particularly preferred commercial product according to the invention is Dow Corning ES-5227 DM from Dow Corning.

Water-in-oil emulsions in which the water-in-oil emulsifier b) is selected only from PEG/PPG-18/18 dimethicone which has an HLB value in the range from 4 to 6.5 and is included in a total amount from 0.2 to 4.0 wt. %, preferably 0.4 to 3.0 wt. % and in particular 0.7 to 2.0 wt. %, relative in each case to the weight of the total propellant-free water-in-oil emulsion, are particularly preferred according to the invention.

The present application preferably provides a cosmetic product consisting of
a water-in-oil emulsion including
a) at least one sweat-inhibiting aluminum salt in a total amount from 5 to 40 wt. %, preferably 10 to 30 wt. %, particularly preferably 15 to 25 wt. % and exceptionally preferably 20 to 23 wt. %, relative in each case to the total weight of the active substance (USP), free from water of crystallization and ligands, in the propellant-free water-in-oil emulsion,
b) at least one water-in-oil emulsifier having an HLB value in the range from 1.5 to 6.5, selected from the group of PEG/PPG-a/b dimethicones, wherein a and b independently of one another denote numbers from 2 to 30, preferably 3 to 24, particularly preferably 12 to 20, in particular 14 to 18, in a total amount from 0.2 to 4.0 wt. %, preferably 0.4 to 3.0 wt. % and in particular 0.7 to 2.0 wt. %, relative in each case to the weight of the total propellant-free water-in-oil emulsion,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17,
d) at least one cosmetic oil that is not a fragrance and not an essential oil,
e) 5-methyl-2-(1-methylethypcyclohexyl-N-ethyl oxamate, preferably (1R,2S,5R)-5-methyl2-(1-methylethyl)cyclohexyl-N-ethyl oxamate,
at least one propellant,
an aerosol dispensing device.

The present application particularly preferably provides a cosmetic product consisting of
a water-in-oil emulsion including
a) at least one sweat-inhibiting aluminum salt in a total amount from 5 to 40 wt. %, preferably 10 to 30 wt. %, particularly preferably 15 to 25 wt. % and exceptionally preferably 20 to 23 wt. %, relative in each case to the total weight of the active substance (USP), free from water of crystallization and ligands, in the propellant-free water-in-oil emulsion,
b) at least one water-in-oil emulsifier having an HLB value in the range from 1.5 to 6.5, selected only from PEG/PPG-18/18 dimethicone which has an HLB value in the range from 4 to 6.5 and is included in a total amount from 0.2 to 4.0 wt. %, preferably 0.4 to 3.0 wt. % and in particular 0.7 to 2.0 wt. %, relative in each case to the weight of the total propellant-free water-in-oil emulsion,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17,
d) at least one cosmetic oil that is not a fragrance and not an essential oil,
e) 5-methyl-2-(1-methyl ethyl)cyclohexyl-N-ethyl oxamate, preferably (1R,2S,5R)-5-methyl2-(1-methylethyl)cyclohexyl-N-ethyl oxamate,
at least one propellant,
an aerosol dispensing device.

The water-in-oil emulsions according to the invention include as the third essential constituent at least one oil-in-water emulsifier or solubilizer having an HLB value in the range from >7 to 20, preferably in the range from >12 to 19, particularly preferably in the range from >14 to 18 and exceptionally preferably in the range from 15 to 17.

The addition of such an oil-in-water emulsifier or solubilizer having an HLB value in the range from >7 to a maximum of 20 has surprisingly proved advantageous for the desired equilibrium between the emulsion stability of the water-in-oil emulsions according to the invention and as fast as possible an active ingredient release of the sweat-inhibiting active ingredient from the internal phase of the emulsion.

Preferred water-in-oil emulsions according to the invention are characterized in that the at least one oil-in-water emulsifier or solubilizer c) has an HLB value >9, preferably an HLB value >12 to 19 and in particular an HLB value >14 to 18. Particularly preferred oil-in-water emulsifiers or solubilizers c) have an HLB value from 15 to 17.

The HLB value is determined according to the invention by the method according to Griffm using the formula: HLB=20*(Mₕ/M), in which Mₕ is the molar mass of the hydrophilic component of a molecule and M is the molar mass of the entire molecule. This gives a scale from 0 to 20.

Preferred water-in-oil emulsions are characterized in that the at least one oil-in-water emulsifier or solubilizer c) is selected from ethoxylated C₈-C₂₄ alkanols having on average 10 to 100 mol of ethylene oxide per mol, ethoxylated C₈-C₂₄ carboxylic acids having on average 10 to 100 mol of ethylene oxide per mol, alkyl mono- and oligoglycosides having 8 to 22 carbon atoms in the alkyl residue and ethoxylated analogs thereof, ethoxylated sterols, partial esters of polyglycerols having 2 to 10 glycerol units and esterified with 1 to 4 saturated or unsaturated, linear or branched, optionally hydroxylated C₈-C₃₀ fatty acid esters, provided that these polyglycerol partial esters have an HLB value in the range from >7 to 20, and mixtures of the aforementioned substances.

The ethoxylated C₈-C₂₄ alkanols have the formula R^{I}O(CH₂CH₂O)ₙH, in which R¹ denotes a linear or branched alkyl and/or alkenyl residue having 8 to 24 carbon atoms and n, the average number of ethylene oxide units per molecule, denotes numbers from 10 to 100, preferably 10 to 30 mol of ethylene oxide.

Particularly preferred according to the invention are adduct compounds of 10 to 100, preferably 10 to 30 mol of ethylene oxide with 1 mol of alkanol, selected from octanol, 2-ethylhexyl alcohol, decanol, lauryl alcohol, isotridecyl alcohol, myristyl alcohol, cetyl alcohol, palmitoleyl alcohol, isocetyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol as well as technical mixtures thereof. Adducts of 10 to 100 mol of ethylene oxide, preferably 10 to 30 mol of ethylene oxide, with 1 mol of alkanol, selected from technical fatty alcohols having 12 to 18 carbon atoms, such as for example coconut, palm, palm kernel or tallow fatty alcohol, are also preferred according to the invention. Isoceteth-20 is exceptionally preferred. The aforementioned ethoxylated alkanols are non-ionic and are also known as polyethylene glycol ethers or non-ionic polyethylene glycol ethers.

The ethoxylated C₈-C₂₄ carboxylic acids have the formula R¹(OCH₂CH₂)ₙOH, in which R¹ denotes a linear or branched, saturated or unsaturated acyl residue having 8-24 carbon atoms and n, the average number of ethylene oxide units per molecule, denotes numbers from 10 to 100, preferably 10 to 50 mol of ethylene oxide, with 1 mol of octanoic acid, 2-ethylhexanoic acid, decanoic acid, lauric acid, isotridecanoic acid, myristic acid, palmitic acid, palmitoleic acid, eicosanoic acid, gadoleic acid, docosanoic acid, erucic acid and brassidic acid and technical mixtures thereof. Adducts of 10 to 100, preferably 10 to 50 mol of ethylene oxide with 1 mol of acid selected from technical fatty acids having 12-18 carbon atoms, such as coconut, palm, palm kernel or tallow fatty acid, are also preferred. PEG-50 monostearate, PEG-100 monostearate, PEG-50 monooleate, PEG-100 monooleate, PEG-50 monolaurate and PEG-100 monolaurate are particularly preferred.

The C₁₂-C₁₈ alkanols having 10 to 30 units of ethylene oxide per molecule and mixtures of these substances are particularly preferred. The C₁₄-C₁₈ alkanols having 20 to 30 units of ethylene oxide per molecule and mixtures of these substances are exceptionally preferred. Isoceteth-12, Isoceteth-20, Isoceteth-30, Isosteareth-12, Isosteareth-20, Isosteareth-30, Laureth-12 and Beheneth-20 are exceptionally preferred in particular.

C₈-C₂₂ alkyl mono- and oligoglycosides are moreover preferably used. C₈-C₂₂ alkyl mono- and oligoglycosides are known, commercial surfactants and emulsifiers. They are produced in particular by reacting glucose or oligosaccharides with primary alcohols having 8-22 carbon atoms. Regarding the glycoside residue, both monoglycosides in which a cyclic sugar residue is glycosidically bound to the fatty alcohol and oligomeric glycosides having a degree of oligomerization of up to around 8, preferably 1 to 2, are suitable. The degree of oligomerization is a statistical mean based on a conventional homolog distribution for technical products. Products available under the trademark Plantacase® include a glucosidically bound C₈-C₁₆ alkyl group at an oligoglucoside residue whose average degree of oligomerization is 1 to 2. Particularly preferred C₈-C₂₂ alkyl mono- and oligoglycosides are selected from octyl glucoside, decyl glucoside, lauryl glucoside, palmityl glucoside, isostearyl glucoside, stearyl glucoside, arachidyl glucoside and behenyl gluco side and mixtures thereof. The acyl glucamides derived from glutamine are also suitable as non-ionic oil-in-water emulsifiers.

Ethoxylated sterols, in particular ethoxylated soy stearols, are also suitable oil-in-water emulsifiers according to the invention. The degree of ethoxylation must be greater than 5, preferably at least 10 in order to have an HLB value greater than 7. Ethoxylated sterols that are suitable as oil-in-water emulsifiers are for example PEG-10 soy sterol, PEG-16 soy sterol and PEG-25 soy sterol.

Partial esters of polyglycerols having 2 to 10 glycerol units and esterified with 1 to 4 saturated or unsaturated linear or branched, optionally hydroxylated C₈-C₃₀ fatty acid esters are also preferably used, provided they have an HLB value in the range from >7 to 20. Diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate and decaglycerol trihydroxystearate are particularly preferred.

The at least one oil-in-water emulsifier or solubilizer having an HLB value in the range from >7 to 20 is preferably selected from non-ionic polyethylene glycol ethers having a corresponding HLB value, i.e. with an HLB value in the range from >7 to 20.

Products that are particularly preferred according to the invention include at least one oil-in-water emulsifier or solubilizer having an HLB value in the range from >7 to 20, preferably in the range from >12 to 19, particularly preferably in the range from >14 to 18 and exceptionally preferably in the range from 15 to 17, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion. Further particularly preferred products include at least one non-ionic polyethylene glycol ether having an HLB value in the range from >7 to 20, preferably in the range from >12 to 19, particularly preferably in the range from >14 to 18 and exceptionally preferably in the range from 15 to, 17, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion. Further particularly preferred products include Isoceteth-20 in a total amount from 0.1 to 2.0 wt. %, preferably 0.2 to 1.2 wt % and particularly preferably 0.5 to 0.8 wt. %, relative in each case to the total propellant-free water-in-oil emulsion.

The present application preferably provides a cosmetic product consisting of
a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, selected from non-ionic polyethylene glycol ethers, in particular Isoceteth-20,
d) at least one cosmetic oil that is not a fragrance and not an essential oil,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate,
at least one propellant,
an aerosol dispensing device.

The present application particularly preferably provides a cosmetic product consisting of
a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, selected from non-ionic polyethylene glycol ethers, in particular Isoceteth-20, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion.
d) at least one cosmetic oil that is not a fragrance and not an essential oil,
e) 5-methyl-2(1-methylethyl)cyclohexyl-N-ethyl oxamate, preferably (1R,2S,5R)-5-methyl-2-(1-methylethyl)cyclohexyl-N-ethyl oxamate,
at least one propellant,
an aerosol dispensing device.

The present application also particularly preferably provides a cosmetic product consisting of
a water-in-oil emulsion including
a) at least one sweat-inhibiting aluminum salt in a total amount from 5 to 40 wt. %, preferably 10 to 30 wt. %, particularly preferably 15 to 25 wt. % and exceptionally preferably 20 to 23 wt. %, relative in each case to the total weight of the active substance (USP), free from water of crystallization and ligands, in the propellant-free water-in-oil emulsion,
b) at least one water-in-oil emulsifier having an HLB value in the range from 1.5 to 6.5 in a total amount from 0.2 to 4.0 wt. %, preferably 0.4 to 3.0 wt. % and in particular 0.7 to 2.0 wt. %, relative in each case to the weight of the total propellant-free water-in-oil emulsion,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, selected from non-ionic polyethylene glycol ethers, in particular Isoceteth-20, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion,
d) at least one cosmetic oil that is not a fragrance and not an essential oil,
e) 5-methyl-2-(1-methylethypcyclohexyl-N-ethyl oxamate, preferably (1R,2S,5R)-5-methyl-2-(1-methylethyl)cyclohexyl-N-ethyl oxamate,
at least one propellant,
an aerosol dispensing device.

The water-in-oil emulsions according to the invention include as the fourth essential constituent at least one cosmetic oil that is not a fragrance and not an essential oil. The cosmetic oil is liquid under normal conditions. Essential oils are understood to be mixtures according to the invention of volatile components which are produced from vegetable raw materials by steam distillation, such as citrus oils for example.

Products that are preferred according to the invention include at least one cosmetic oil that is not a fragrance and not an essential oil in a total amount from 1 to 30 wt. %, preferably 5 to 26 wt. %, particularly preferably 9 to 24 wt. %, exceptionally preferably 15 to 20 wt. %, relative in each case to the weight of the total propellant-free water-in-oil emulsion.

A distinction is made in cosmetic oils between volatile and non-volatile oils. Non-volatile oils are understood to be oils that at 20° C. and under an ambient pressure of 1013 hPa have a vapor pressure of less than 2.66 Pa (0.02 mm Hg). Volatile oils are understood to be oils that at 20° C. and under an ambient pressure of 1013 hPa have a vapor pressure of 2.66 Pa to 40,000 Pa (0.02 mm to 300 mm Hg), preferably 10 to 12,000 Pa (0.1 to 90 mm Hg), particularly preferably 13 to 3000 Pa, exceptionally preferably 15 to 500 Pa.

Volatile cosmetic oils are conventionally selected from cyclic silicone oils having the INCI name cyclomethicones. The INCI name cyclomethicones is understood to mean in particular cyclotrisiloxane (hexamethyl cyclotrisiloxane), cyclotetrasiloxane (octamethyl cyclotetrasiloxane), cyclopentasiloxane (decamethyl cyclopentasiloxane) and cyclohexasiloxane (dodecamethyl cyclohexasiloxane). These oils have a vapor pressure of approx. 13 to 15 Pa at 20° C.

Cyclomethicones are known in the prior art as being very suitable oils for cosmetic products, in particular for sweat-inhibiting and deodorizing products such as sprays and sticks. Owing to their persistence in the environment, however, it can be preferable according to the invention to avoid the use of cyclomethicones. In a specially preferred embodiment the products according to the invention include a maximum of 1 wt. %, preferably a maximum of 0.1 wt. % of cyclomethicones, relative in each case to the weight of the water-in-oil emulsion, without taking into account the weight of the propellant.

A cyclomethicone substitute that is preferred according to the invention is a mixture of C₁₃-C₁₆ isoparaffins, C₁₂-C₁₄ isoparaffins and C₁₃-C₁₅ alkanes, whose viscosity at 25° C. is in the range from 2 to 6 mPas and which have a vapor pressure at 20° C. in the range from 10 to 150 Pa, preferably 100 to 150 Pa. Such a mixture is obtainable for example under the name SiClone SR-5 from Presperse Inc.

Further preferred volatile silicone oils are selected from volatile linear silicone oils, in particular volatile linear silicone oils having 2 to 10 siloxane units, such as hexamethyldisiloxane (L₂), octamethyltrisiloxane (L₃), decamethyltetrasiloxane (L₄), such as are included for example in the commercial products DC 2-1184, Dow Coming® 200 (0.65 cSt) and Dow Corning® 200 (1.5 cSt) from Dow Corning, and low-molecular-weight phenyl trimethicone having a vapor pressure at 20° C. of approximately 2000 Pa, such as is obtainable for example from GE Bayer Silicones/Momentive under the name Baysilone Fluid PD 5.

In the interests of a drier skin feel and faster active substance release, preferred antiperspirant compositions according to the invention include at least one volatile silicone oil, which can be cyclic or linear.

In the interests of a drier skin feel and faster active substance release, further preferred products according to the invention include at least one volatile non-silicone oil. Preferred volatile non-silicone oils are selected from C₈-C₁₆ isoparaffins, in particular from isononane, isodecane, isoundecane, isododecane, isotridecane, isotetradecane, isopentadecane and isohexadecane, as well as mixtures thereof. C₁₀-C₁₃ isoparaffin mixtures are preferred, in particular those having a vapor pressure at 20° C. of approximately 10 to 400 Pa, preferably 13 to 100 Pa. This at least one C₈-C₁₆ isoparaffin is preferably included in a total amount from 1 to 30 wt. %, preferably 3 to 26 wt. %, particularly preferably 5 to 24 wt. %, exceptionally preferably 8 to 17 wt. %, relative in each case to the weight of the water-in-oil emulsion, without taking into account the weight of the propellant.

Further preferred water-in-oil emulsions according to the invention include at least one non-volatile cosmetic oil selected from non-volatile silicone oils and non-volatile non-silicone oils. Residues of the dissolved antiperspirant active ingredient and also residues of constituents that are insoluble in the emulsion, such as talc and silica particles, can be successfully masked with a non-volatile oil. Furthermore, with a mixture of various oils, in particular non-volatile and volatile oil, parameters such as skin feel, residue visibility and stability of the composition according to the invention can be fine-tuned and better adjusted to the requirements of consumers.

Preferred products according to the invention are characterized in that the cosmetic oil d) comprises at least one volatile oil having a vapor pressure from 10 to 3000 Pa at 20° C., in a total amount from 10 to 100 wt. %, particularly preferably 30 to 80 wt. %, relative in each case to the total weight of the cosmetic oils d).

It is of course likewise possible to formulate products according to the invention with a low content of volatile oils, i.e. with 0.5 to 15 wt. % of volatile oils, relative to the weight of the propellant-free water-in-oil emulsion, or even without volatile oils.

Particularly preferred according to the invention as the cosmetic oil d) are moreover esters of linear or branched saturated or unsaturated fatty alcohols having 2 to 30 carbon atoms with linear or branched saturated or unsaturated fatty acids having 2 to 30 carbon atoms, which can be hydroxylated. Esters of linear or branched saturated fatty alcohols having 2 to 5 carbon atoms with linear or branched saturated or unsaturated fatty acids having 10 to 18 carbon atoms, which can be hydroxylated, are preferred. Preferred examples thereof are isopropyl palmitate, isopropyl stearate, isopropyl myristate, 2-hexyldecyl stearate, 2-hexyldecyl laurate, isodecyl neopentanoate, isononyl isononanoate, 2-ethylhexyl palmitate and 2-ethylhexyl stearate. Likewise preferred are isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyl dodecylpalmitate, butyl octanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate, ethylene glycol dipalmitate. n-Hexyl laurate, n-decyl oleate, oleyl oleate, oleyl erucate, erucyl oleate, C₁₂-C₁₅ alkyl lactate and di-C₁₂-C₁₃ alkyl malate and the benzoic acid esters of linear or branched C₈-C₂₂ alkanols. Benzoic acid C₁₂-C₁₅ alkyl esters, available for example as the commercial product Finsolv® TN (C₁₂-C₁₅ alkyl benzoate), benzoic acid isostearyl esters, available for example as Finsolv® SB, 2-ethylhexyl benzoate, available for example as Finsolv® EB, and benzoic acid 2-octyl dodecyl esters, available for example as Finsolv® BOD, are particularly preferred.

The use of isopropyl esters of C₁₂-C₁₈ carboxylic acids, in particular the use of isopropyl myristate and particularly preferably mixtures of isopropyl myristate with C₁₀-C₁₃ isoparaffin mixtures, the latter preferably having a vapor pressure at 20° C. of 10 to 400 Pa, has proved particularly advantageous for emulsion stability and active ingredient release.

Triethyl citrate is a further particularly preferred ester oil. Further products that are preferred according to the invention include triethyl citrate and at least one C₈-C₁₆ isoparaffin, selected from isononane, isodecane, isoundecane, isododecane, isotridecane, isotetradecane, isopentadecane and isohexadecane as well as mixtures of these isoparaffins. Further products that are preferred according to the invention include triethyl citrate and at least one C₈-C₁₆ isoparaffin, selected from isononane, isodecane, isoundecane, isododecane, isotridecane as well as mixtures of these C₈-C₁₆ isoparaffins. Further products that are preferred according to the invention include triethyl citrate and a mixture of isodecane, isoundecane, isododecane and isotridecane.

Water-in-oil emulsions that are preferred according to the invention include at least one ester of linear or branched saturated or unsaturated fatty alcohols having 2 to 30 carbon atoms with linear or branched saturated or unsaturated fatty acids having 2 to 30 carbon atoms, which can be hydroxylated, in a total amount from 1 to 30 wt. %, preferably 5 to 26 wt. %, particularly preferably 9 to 24 wt. %, exceptionally preferably 12 to 17 wt. %, relative in each case to the weight of the total propellant-free water-in-oil emulsion.

Further non-volatile non-silicone oils that are preferred according to the invention are selected from branched saturated or unsaturated fatty alcohols having 6 to 30 carbon atoms. These alcohols are frequently also referred to as Guerbet alcohols, as they are obtainable by the Guerbet reaction. Preferred alcohol oils are 2-hexyl decanol, 2-octyl dodecanol and 2-ethylhexyl alcohol. Isostearyl alcohol is likewise preferred. Further preferred non-volatile oils are selected from mixtures of Guerbet alcohols and Guerbet alcohol esters, for example 2-hexyl decanol and 2-hexyl decyl laurate.

The expression “triglyceride” as used below means “glycerol triester”. Further non-volatile oils that are preferred according to the invention are selected from the triglycerides of linear or branched, saturated or unsaturated, optionally hydroxylated C₈-₃0 fatty acids, provided they are liquid under normal conditions. The use of natural oils, for example soybean oil, cottonseed oil, sunflower oil, palm oil, palm kernel oil, linseed oil, almond oil, castor oil, corn oil, rapeseed oil, olive oil, sesame oil, thistle oil, wheat germ oil, peach kernel oil and the liquid components of coconut butter and the like, can be particularly suitable. Synthetic triglyceride oils, in particular capric/caprylic triglycerides, for example the commercial products Myritol® 318 or Myritol® 331 (BASF/Cognis) with unbranched fatty acid residues, and also glyceryl triisostearol and glyceryl tri(2-ethylhexanoate) with branched fatty acid residues are particularly preferred. Such triglyceride oils preferably make up a proportion of less than 50 wt. % of the total weight of all cosmetic oils in the product according to the invention. The total weight of triglyceride oils is particularly preferably 0.5 to 10 wt. %, preferably 1 to 5 wt. %, relative in each case to the weight of the total water-in-oil emulsion, without taking into account the weight of the propellant.

Further non-volatile non-silicone oils that are particularly preferred according to the invention are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Further non-volatile non-silicone oils that are particularly preferred according to the invention are selected from the symmetrical, asymmetrical or cyclic esters of carbonic acid with C₆-C₂₀ alcohols, for example di-n-caprylyl carbonate (Cetiol® CC) or di-(2-ethylhexyl)carbonate (Tegosoft DEC). By contrast, esters of carbonic acid with C₁-C₅ alcohols, for example glycerol carbonate or propylene carbonate, are not compounds that are suitable as the cosmetic oil.

Further oils that can be preferred according to the invention are selected from the esters of dimers of unsaturated C₁₂-C₂₂ fatty acids (dimer fatty acids) with monohydric linear, branched or cyclic C₂-C₁₈ alkanols or with polyhydric linear or branched C₂-C₆ alkanols. The total weight of dimer fatty acids is particularly preferably 0.5 to 10 wt. %, preferably 1 to 5 wt. %, relative in each case to the weight of the total water-in-oil emulsion, without taking into account the weight of the propellant.

Further cosmetic oils that are particularly preferred according to the invention are selected from non-volatile silicone oils. Non-volatile silicone oils that are preferred according to the invention are selected from linear polyalkyl siloxanes having a kinematic viscosity at 25° C. of at least 5 cSt to 2000 cSt, in particular selected from linear polydimethyl siloxanes having a kinematic viscosity at 25° C. of 5 cSt to 2000 cSt, preferably 10 to 350 cSt, particularly preferably 50 to 100 cSt, such as are obtainable for example under the trade names Dow Corning® 200 and Xiameter PMX from Dow Corning and Xiameter respectively. Further preferred non-volatile silicone oils are phenyl trimethicones having a kinematic viscosity at 25° C. of 10 to 100 cSt, preferably 15 to 30 cSt, and cetyl dimethicones.

Water-in-oil emulsions that are preferred according to the invention include at least one non-volatile silicone oil, which is preferably selected from linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, in a total amount from 0.1 to 30 wt. %, preferably 1 to 24 wt. %, particularly preferably 2 to 18 wt. %, exceptionally preferably 4 to 10 wt. %, relative in each case to the weight of the total propellant-free water-in-oil emulsion.

With regard to the desired equilibrium between emulsion stability of the water-in-oil emulsions according to the invention and the as rapid release as possible of the sweat-inhibiting active ingredient from the internal phase of the emulsion on the one hand and the stabilizing of the cooling active ingredient according to the invention 5-methyl-2-(1-methylethyl)cyclohexyl-N-ethyl oxamate on the other, the use of cosmetic oils d) that are selected from mixtures of C₈-C₁₆ isoparaffins and esters of the linear or branched saturated or unsaturated fatty alcohols having 2 to 30 carbon atoms with linear or branched saturated or unsaturated, optionally hydroxylated fatty acids having 2 to 30 carbon atoms, mixtures of C₈-C₁₆ isoparaffins and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, as well as mixtures of C₈-C₁₆ isoparaffins and esters of linear or branched saturated or unsaturated fatty alcohols having 2 to 30 carbon atoms with linear or branched saturated or unsaturated, optionally hydroxylated fatty acids having 2 to 30 carbon atoms and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, have surprisingly proved advantageous.

The present application particularly preferably provides a cosmetic product consisting of
a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, selected from non-ionic polyethylene glycol ethers, in particular Isoceteth-20, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion.
d) at least one cosmetic oil that is not a fragrance and not an essential oil in a total amount from 1 to 30 wt. %, preferably 5 to 26 wt. %, particularly preferably 9 to 24 wt. %, exceptionally preferably 15 to 20 wt. %, relative in each case to the weight of the total propellant-free water-in-oil emulsion,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate,
at least one propellant,
an aerosol dispensing device.

The present application preferably also provides a cosmetic product consisting of
a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17,
d) at least one cosmetic oil that is not a fragrance and not an essential oil and that is selected from C₈-C₁₆ isoparaffins, esters of linear or branched saturated or unsaturated fatty alcohols having 2 to 30 carbon atoms with linear or branched saturated or unsaturated, optionally hydroxylated fatty acids having 2 to 30 carbon atoms and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, as well as mixtures of the aforementioned oils,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate,
at least one propellant,
an aerosol dispensing device.

The present application particularly preferably provides a cosmetic product consisting of
a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, selected from non-ionic polyethylene glycol ethers, in particular Isoceteth-20, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion.
d) at least one cosmetic oil that is not a fragrance and not an essential oil and that is selected from C₈-C₁₆ isoparaffins, esters of linear or branched saturated or unsaturated fatty alcohols having 2 to 30 carbon atoms with linear or branched saturated or unsaturated, optionally hydroxylated fatty acids having 2 to 30 carbon atoms and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, as well as mixtures of the aforementioned oils, in a total content of 1 to 30 wt. %, preferably 5 to 26 wt. %, particularly preferably 9 to 24 wt. %, exceptionally preferably 15 to 20 wt. %, relative in each case to the weight of the total propellant-free water-in-oil emulsion,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate,
at least one propellant,
an aerosol dispensing device.

The present application also particularly preferably provides a cosmetic product consisting of
a water-in-oil emulsion including
a) at least one sweat-inhibiting aluminum salt in a total amount from 5 to 40 wt. %, preferably 10 to 30 wt. %, particularly preferably 15 to 25 wt. % and exceptionally preferably 20 to 23 wt. %, relative in each case to the total weight of the active substance (USP), free from water of crystallization and ligands, in the propellant-free water-in-oil emulsion,
b) at least one water-in-oil emulsifier having an HLB value in the range from 1.5 to 6.5 in a total amount from 0.2 to 4.0 wt. %, preferably 0.4 to 3.0 wt. % and in particular 0.7 to 2.0 wt. %, relative in each case to the weight of the total propellant-free water-in-oil emulsion,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, selected from non-ionic polyethylene glycol ethers, in particular Isoceteth-20, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion,
d) at least one cosmetic oil that is not a fragrance and not an essential oil and that is selected from mixtures of C₈-C₁₆ isoparaffins and esters of linear or branched saturated or unsaturated fatty alcohols having 2 to 30 carbon atoms with linear or branched saturated or unsaturated, optionally hydroxylated fatty acids having 2 to 30 carbon atoms and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, in a total amount from 1 to 30 wt. %, preferably 5 to 26 wt. %, particularly preferably 9 to 24 wt. %, exceptionally preferably 15 to 20 wt. %, relative in each case to the weight of the total propellant-free water-in-oil emulsion,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate,
at least one propellant,
an aerosol dispensing device.

It was moreover possible to lower the cyclomethicone content of products according to the invention without adversely affecting the product properties. Preferred products according to the invention therefore include, relative to the total propellant-free water-in-oil emulsion, less than 5.0 wt. % of cyclomethicone, preferably less than 3.0 wt. % of cyclomethicone and particularly preferably less than 1.0 wt. % of cyclomethicone. Most particularly preferred products according to the invention are free from cyclomethicones.

The products according to the invention include as the fifth essential constituent the cooling active ingredient 5-methyl-2-(1-methylethyl)cyclohexyl-N-ethyl oxamate. The isomer (1R,2S,5R)-5-methyl-2-(1-methylethyl)cyclohexyl-N-ethyl oxamate is preferably included. This cooling active ingredient combines a long-lasting cooling effect even in low usage concentrations with a low inherent odor, a high skin compatibility and a good release, in particular from a water-in-oil emulsion. Preferred products according to the invention include 5-methyl-2-(1-methylethyl)cyclohexyl-N-ethyl oxamate, preferably (1R,2S,5R)-5-methyl-2-(1-methylethypcyclohexyl-N-ethyl oxamate, in a total amount from 0.05 to 2 wt. %, preferably 0.1 to 1.5 wt. %, particularly preferably 0.2 to 1 wt. %, exceptionally preferably 0.3 to 0.5 wt. %, wherein all stated amounts relate to the weight of the water-in-oil emulsion, without taking into account the weight of the propellant.

“Long-lasting” according to the invention is understood to mean a time of at least one hour, preferably two to at least four hours, following application of the product onto the skin.

The present application preferably also provides a cosmetic product consisting of
a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17,
d) at least one cosmetic oil that is not a fragrance and not an essential oil and that is selected from C₈-C₁₆ isoparaffins, esters of linear or branched saturated or unsaturated fatty alcohols having 2 to 30 carbon atoms with linear or branched saturated or unsaturated, optionally hydroxylated fatty acids having 2 to 30 carbon atoms and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, as well as mixtures of the aforementioned oils,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate, in a total amount from 0.05 to 2 wt. %, preferably 0.1 to 1.5 wt. %, particularly preferably 0.2 to 1 wt. %, exceptionally preferably 0.3 to 0.5 wt. %, wherein all stated amounts relate to the weight of the water-in-oil emulsion, without taking into account the weight of the propellant,
at least one propellant,
an aerosol dispensing device.

The present application particularly preferably provides a cosmetic product consisting of
a water-in-oil emulsion including
a) at least one sweat-inhibiting aluminum salt,
b) at least one water-in-oil emulsifier having an EILB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, selected from non-ionic polyethylene glycol ethers, in particular Isoceteth-20, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion,
d) at least one cosmetic oil that is not a fragrance and not an essential oil and that is selected from C₈-C₁₆ isoparaffins, esters of linear or branched saturated or unsaturated fatty alcohols having 2 to 30 carbon atoms with linear or branched saturated or unsaturated, optionally hydroxylated fatty acids having 2 to 30 carbon atoms and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, as well as mixtures of the aforementioned oils, in a total amount from 1 to 30 wt. %, preferably 5 to 26 wt. %, particularly preferably 9 to 24 wt. %, exceptionally preferably 15 to 20 wt. %, relative in each case to the weight of the total propellant-free water-in-oil emulsion,
e) 5-methyl-2-(1-methylethypcyclohexyl-N-ethyl oxamate, preferably (1R,2S,5R)-5-methyl-2-(1-methylethyl)cyclohexyl-N-ethyl oxamate, in a total amount from 0.05 to 2 wt. %, preferably 0.1 to 1.5 wt. %, particularly preferably 0.2 to 1 wt. %, exceptionally preferably 0.3 to 0.5 wt. %, wherein all stated amounts relate to the weight of the water-in-oil emulsion, without taking into account the weight of the propellant,
at least one propellant,
an aerosol dispensing device.

The present application also particularly preferably provides a cosmetic product consisting of
a water-in-oil emulsion including
a) at least one sweat-inhibiting aluminum salt in a total amount from 5 to 40 wt. %, preferably 10 to 30 wt. %, particularly preferably 15 to 25 wt. % and exceptionally preferably 20 to 23 wt. %, relative in each case to the total weight of the active substance (USP), free from water of crystallization and ligands, in the propellant-free water-in-oil emulsion,
b) at least one water-in-oil emulsifier having an HLB value in the range from 1.5 to 6.5 in a total amount from 0.2 to 4.0 wt. %, preferably 0.4 to 3.0 wt. % and in particular 0.7 to 2.0 wt. %, relative in each case to the weight of the total propellant-free water-in-oil emulsion,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, selected from non-ionic polyethylene glycol ethers, in particular Isoceteth-20, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion.
d) at least one cosmetic oil that is not a fragrance and not an essential oil and that is selected from mixtures of C₈-C₁₆ isoparaffins and esters of linear or branched saturated or unsaturated fatty alcohols having 2 to 30 carbon atoms with linear or branched saturated or unsaturated, optionally hydroxylated fatty acids having 2 to 30 carbon atoms and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, in a total amount from 1 to 30 wt. %, preferably 5 to 26 wt. %, particularly preferably 9 to 24 wt. %, exceptionally preferably 15 to 20 wt. %, relative in each case to the weight of the total propellant-free water-in-oil emulsion,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate, in a total amount from 0.05 to 2 wt. %, preferably 0.1 to 1.5 wt. %, particularly preferably 0.2 to 1 wt. %, exceptionally preferably 0.3 to 0.5 wt. %, wherein all stated amounts relate to the weight of the water-in-oil emulsion, without taking into account the weight of the propellant,
at least one propellant,
an aerosol dispensing device.

Preferred products according to the invention include at least one fragrance. The definition of a fragrance within the meaning of the present application agrees with the conventional technical definition in the art, as can be taken from RÖMPP Chemie Lexikon, issued December 2007. According to this definition, a fragrance is a chemical compound having an odor and/or flavor, which excites the receptors of the hair cells (adequate stimulus). The necessary physical and chemical properties are a low molar mass of a maximum of 300 g/mol, a high vapor pressure, minimal water solubility, high lipid solubility as well as weak polarity and the presence of at least one osmophore group in the molecule. To delimit volatile, low-molecular-weight substances which conventionally and within the meaning of the present application are regarded and used not as a fragrance but primarily as a solvent, such as for example ethanol, propanol, isopropanol and acetone, from fragrances according to the invention, fragrances according to the invention have a molar mass from 74 to 300 g/mol, include at least one osmophore group in the molecule and have an odor and/or a flavor, in other words they excite the receptors of the hair cells of the olfactory system.

Perfumes, perfume oils, perfume oil constituents or individual fragrance compounds can be used to perfume the products according to the invention. According to the invention perfume oils or fragrances can be individual fragrance compounds, for example the synthetic products of the ester, ether, aldehyde, ketone, alcohol and hydrocarbon type. Fragrance compounds of the ester type are for example benzyl acetate, phenoxyethyl isobutyrate, p-tert-butyl cyclohexyl acetate, linalyl acetate, dimethyl benzyl carbinyl acetate (DMBCA), phenylethyl acetate, benzyl acetate, ethyl methyl phenyl glycinate, allyl cyclohexyl propionate, styrallyl propionate, benzyl salicylate, cyclohexyl salicylate, floramat, melusat and jasmecyclate. The ethers include for example benzyl ethyl ethers and ambroxan, the aldehydes include for example linear alkanals having 8 to 18 C atoms, citral, citronellal, citronellyl oxyacetaldehyde, cyclamen aldehyde, lilial and bourgeonal, the ketones include for example ionones, alpha-isomethyl ionone and methyl cedryl ketone, the alcohols include anethol, citronellol, eugenol, geraniol, linalool, phenyl ethyl alcohol, alpha-terpineol, beta-terpineol, gamma-terpineol and delta-terpineol, the hydrocarbons primarily include terpenes such as limonene and pinene. Mixtures of different fragrances which together generate an attractive scent note are preferably used, however.

Such perfume oils can also include natural fragrance mixtures, such as are obtainable from plant sources, for example pine, citrus, jasmine, patchouli, rose or ylang-ylang oil. Likewise suitable are muscatel sage oil, chamomile oil, clove oil, melissa oil, mint oil, cinnamon leaf oil, lime blossom oil, juniper berry oil, vetiver oil, olibanum oil, galbanum oil and laudanum oil as well as orange blossom oil, neroli oil, orange peel oil and sandalwood oil.

In order to be perceptible, a fragrance must be volatile, wherein in addition to the nature of the functional groups and the structure of the chemical compound, the molar mass also plays an important role. Thus most fragrances have molar masses of up to approx. 200 daltons, whereas molar masses of 300 daltons and above constitute an exception. Owing to the differing volatility of fragrances, the odor of a perfume or fragrance composed of a plurality of fragrances changes as it evaporates, wherein the odor impressions are divided into “top note”, “middle note” or “body”, and “end note” or “dry-out”. As the odor perception is also based to a great extent on the odor intensity, the top note of a perfume or fragrance does not consist solely of highly volatile compounds, whereas the end note consists largely of less volatile, i.e. fixative, fragrances. When composing perfumes, more highly volatile fragrances can be bound for example to certain fixatives, thus preventing their too rapid evaporation. Thus the following categorization of fragrances into “more highly volatile” or “fixative” fragrances says nothing about the odor impression or whether the corresponding fragrance is perceived as a top note or middle note.

Fixative perfumes which can be used in the context of the present invention are for example essential oils such as angelica root oil, aniseed oil, arnica flower oil, basil oil, bay oil, bergamot oil, champaca flower oil, noble fir oil, noble fir cone oil, elemi oil, eucalyptus oil, fennel oil, spruce needle oil, galbanum oil, geranium oil, gingergrass oil, guaiac wood oil, gurjun balsam oil, helichrysum oil, hon-sho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemongrass oil, lime oil, mandarin oil, melissa oil, musk seed oil, myrrh oil, clove oil, neroli oil, niaouli oil, olibanum oil, orange oil, origanum oil, palmarosa oil, patchouli oil, Peru balsam oil, petitgrain oil, pepper oil, peppermint oil, pimento oil, pine oil, rose oil, rosemary oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil and cypress oil. However, higher-boiling or solid fragrances of natural or synthetic origin can also be used in the context of the present invention as fixative fragrances or fragrance blends, i.e. as fragrances. These compounds include the compounds listed below and mixtures thereof: ambrettolide, allyl acetate, alpha-amylcinnamaldehyde, anethol, anisaldehyde, anisic alcohol, anisol, methyl anthranilate, acetophenone, benzyl acetone, benzaldehyde, ethyl benzoate, benzophenone, benzyl alcohol, benzyl acetate, benzyl benzoate, benzyl formate, benzyl valerianate, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, methyl heptine carboxylate, heptaldehyde, hydroquinone dimethyl ether, hydroxycinnamaldehyde, hydroxycinnamic alcohol, indole, irone, isoeugenol, isoeugenol methyl ether, isosafrole, jasmone, camphor, carvacrol, carvone, p-cresol methyl ether, coumarin, p-methoxyacetophenone, methyl-n-amyl ketone, methyl methyl anthranilate, p-methyl acetophenone, methyl chavicol, p-methyl quinoline, methyl-β-naphthyl ketone, methyl-n-nonyl acetaldehyde, methyl-n-nonyl ketone, muscone, β-naphthol ethyl ether, β-naphthol methyl ether, nerol, nitrobenzene, n-nonyl aldehyde, nonyl alcohol, n-octyl aldehyde, p-oxyacetophenone, pentadecanolide, β-phenyl ethyl alcohol, phenyl acetaldehyde dimethyl acetal, phenyl acetic acid, pulegone, safrole, isoamyl salicylate, methyl salicylate, hexyl salicylate, cyclohexyl salicylate, santalol, skatole, alpha-terpineol, beta-terpineol, gamma-terpineol, delta-terpineol, thymene, thymol, gamma-undelactone, vanillin, veratrum aldehyde, cinnamaldehyde, cinnamyl alcohol, cinnamic acid, ethyl cinnamate, benzyl cinnamate.

The more highly volatile fragrances include in particular the lower-boiling fragrances of natural or synthetic origin, which can be used alone or in mixtures. Examples of more highly volatile fragrances are alkyl isothiocyanates (alkyl mustard oils), butanedione, limonene, linalool, linalyl acetate, linalyl propionate, menthol, menthone, methyl-n-heptenone, phellandrene, phenylacetaldehyde, terpinyl acetate, citral, citronellal.

Particularly preferred products according to the invention include at least one fragrance in a total amount from 0.00001 to 10 wt. %, preferably 0.5 to 7 wt. %, particularly preferably 2 to 5 wt. %, relative in each case to the total propellant-free water-in-oil emulsion.

In a further preferred embodiment the water-in-oil emulsions according to the invention include at least one polyol selected from the polyhydric C₂-C₉ alkanols having 2 to 6 hydroxyl group and polyethylene glycols having 3 to 20 ethylene oxide units and mixtures thereof. Particularly preferred water-in-oil emulsions according to the invention include at least one polyhydric C₂-C₉ alkanol having 2 to 6 hydroxyl groups and/or at least one polyethylene glycol having 3 to 20 ethylene oxide units.

Preferred products according to the invention are characterized in that the water-in-oil emulsion moreover includes at least one polyhydric C₂-C₉ alkanol having 2 to 6 hydroxyl groups and/or at least one polyethylene glycol having 3 to 20 ethylene oxide units, selected from 1,2-propylene glycol, 1,3-propylene glycol, 2-methyl-1,3-propanediol, glycerol, 1,2-butylene glycol, 1,3-butylene glycol, 1,4-butylene glycol, pentylene glycols such as 1,2-pentanediol and 1,5-pentanediol, hexanediols such as 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol, cis-1,4-dimethylol cyclohexane, trans-1,4-dimethylol cyclohexane, any isomer mixtures of cis- and trans-1,4-dimethylol cyclohexane as well as mixtures of the aforementioned substances. These substances have a positive influence on the residue behavior, the emulsion stability and a stable sprayability of the water-in-oil emulsions according to the invention. A particularly preferred polyhydric C₂-C₉ having 2 to 6 hydroxyl groups alkanol in this regard is 1,2-propylene glycol.

The proportion by weight of the polyhydric C₂-C₉ alkanol having 2 to 6 hydroxyl groups and/or of the polyethylene glycol having 3 to 20 ethylene oxide units, relative to the total content of these substances in the propellant-free water-in-oil emulsion, is by preference 0.5 to 35 wt. %, preferably 2.0 to 30 wt. %, particularly preferably 5 to 25 wt. %, more preferably 10 to 23 wt. % or 12 to 18 wt. %.

The present application preferably also provides a cosmetic product consisting of
a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17,
d) at least one cosmetic oil that is not a fragrance and not an essential oil and that is selected from C₆-C₁₆ isoparaffins, esters of linear or branched saturated or unsaturated fatty alcohols having 2 to 30 carbon atoms with linear or branched saturated or unsaturated, optionally hydroxylated fatty acids having 2 to 30 carbon atoms and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, as well as mixtures of the aforementioned oils,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate, in a total amount from 0.05 to 2 wt. %, preferably 0.1 to 1.5 wt. %, particularly preferably 0.2 to 1 wt. %, exceptionally preferably 0.3 to 0.5 wt. %, wherein all stated amounts relate to the weight of the water-in-oil emulsion, without taking into account the weight of the propellant,
f) at least one polyhydric C₂-C₉ alkanol having 2 to 6 hydroxyl groups and/or at least one polyethylene glycol having 3 to 20 ethylene oxide units, preferably selected from 1,2-propylene glycol, in a total amount from 0.5 to 35 wt. %, preferably 2.0 to 30 wt. %, particularly preferably 5 to 25 wt. %, more preferably 10 to 23 wt. % or 12 to 18 wt. %, all stated amounts relating to the weight of the water-in-oil emulsion, without taking into account the weight of the propellant,
at least one propellant,
an aerosol dispensing device.

The present application particularly preferably provides a cosmetic product consisting of
a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, selected from non-ionic polyethylene glycol ethers, in particular Isoceteth-20, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion,
d) at least one cosmetic oil that is not a fragrance and not an essential oil and that is selected from C₈-C₁₆ isoparaffins, esters of linear or branched saturated or unsaturated fatty alcohols having 2 to 30 carbon atoms with linear or branched saturated or unsaturated, optionally hydroxylated fatty acids having 2 to 30 carbon atoms and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, as well as mixtures of the aforementioned oils, in a total amount from 1 to 30 wt. %, preferably 5 to 26 wt. %, particularly preferably 9 to 24 wt. %, exceptionally preferably 15 to 20 wt. %, relative in each case to the weight of the total propellant-free water-in-oil emulsion,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate, in a total amount from 0.05 to 2 wt. %, preferably 0.1 to 1.5 wt. %, particularly preferably 0.2 to 1 wt. %, exceptionally preferably 0.3 to 0.5 wt. %, wherein all stated amounts relate to the weight of the water-in-oil emulsion, without taking into account the weight of the propellant,
f) at least one polyhydric C₂-C₉ alkanol having 2 to 6 hydroxyl groups and/or at least one polyethylene glycol having 3 to 20 ethylene oxide units, preferably selected from 1,2-propylene glycol, in a total amount from 0.5 to 35 wt. %, preferably 2.0 to 30 wt. %, particularly preferably 5 to 25 wt. %, more preferably 10 to 23 wt. % or 12 to 18 wt. %, all stated amounts relating to the weight of the water-in-oil emulsion, without taking into account the weight of the propellant,
at least one propellant,
an aerosol dispensing device.

The present application also particularly preferably provides a cosmetic product consisting of
a water-in-oil emulsion including
a) at least one sweat-inhibiting aluminum salt in a total amount from 5 to 40 wt. %, preferably 10 to 30 wt. %, particularly preferably 15 to 25 wt. % and exceptionally preferably 20 to 23 wt. %, relative in each case to the total weight of the active substance (USP), free from water of crystallization and ligands, in the propellant-free water-in-oil emulsion,
b) at least one water-in-oil emulsifier having an HLB value in the range from 1.5 to 6.5 in a total amount from 0.2 to 4.0 wt. %, preferably 0.4 to 3.0 wt. % and in particular 0.7 to 2.0 wt. %, relative in each case to the weight of the total propellant-free water-in-oil emulsion,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17, selected from non-ionic polyethylene glycol ethers, in particular Isoceteth-20, in a total amount from 0.1 to 2.0 wt. %, preferably 0.2 to 1.2 wt. % and particularly preferably 0.5 to 0.8 wt. %, relative in each case to the weight of the total propellant-free water-in-oil emulsion.
d) at least one cosmetic oil that is not a fragrance and not an essential oil and that is selected from mixtures of C₈-C₁₆ isoparaffins and esters of linear or branched saturated or unsaturated fatty alcohols having 2 to 30 carbon atoms with linear or branched saturated or unsaturated, optionally hydroxylated fatty acids having 2 to 30 carbon atoms and linear polyalkyl siloxanes having a kinematic viscosity at 25° C. of 5 cSt to 2000 cSt, preferably 10 to 350 cSt, particularly preferably 50 to 100 cSt, in particular selected from linear polydimethyl siloxanes having a kinematic viscosity at 25° C. of 5 cSt to 2000 cSt, preferably 10 to 350 cSt, particularly preferably 50 to 100 cSt, in a total amount from 1 to 30 wt. %, preferably 5 to 26 wt. %, particularly preferably 9 to 24 wt. %, exceptionally preferably 15 to 20 wt. %, relative in each case to the weight of the total propellant-free water-in-oil emulsion,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate, in a total amount from 0.05 to 2 wt. %, preferably 0.1 to 1.5 wt. %, particularly preferably 0.2 to 1 wt. %, exceptionally preferably 0.3 to 0.5 wt. %, wherein all stated amounts relate to the weight of the water-in-oil emulsion, without taking into account the weight of the propellant,
f) at least one polyhydric C₂-C₉ alkanol having 2 to 6 hydroxyl groups and/or at least one polyethylene glycol having 3 to 20 ethylene oxide units, preferably selected from 1,2-propylene glycol, in a total amount from 0.5 to 35 wt. %, preferably 2.0 to 30 wt. %, particularly preferably 5 to 25 wt. %, more preferably 10 to 23 wt. % or 12 to 18 wt. %, all stated amounts relating to the weight of the water-in-oil emulsion, without taking into account the weight of the propellant,
at least one propellant,
an aerosol dispensing device.

On the basis of the combinations according to the invention it is possible to lower the ethanol content of products according to the invention without adversely affecting the product properties. Preferred products according to the invention therefore include, relative to the total propellant-free water-in-oil emulsion, less than 4.0 wt. % of ethanol, preferably less than 3.0 wt. % of ethanol and particularly preferably less than 1.0 wt. % of ethanol. Most particularly preferred products according to the invention are free from ethanol.

The products according to the invention include water preferably in a total amount from 5 to 55 wt. %, preferably 10 to 40 wt. %, particularly preferably 15 to 30 wt. %, exceptionally preferably 20 to 25 wt. %, relative in each case to the weight of the total propellant-free water-in-oil emulsion.

To further optimize the cooling effect of the products according to the invention and for example to achieve a very strong effect (“flash”) on the skin shortly after applying the product, which is then further extended by 5-methyl-2-(1-methylethyl)cyclohexyl-N-ethyl oxamate, preferably (1R,2S,5R)-5-methyl-2-(1-methylethyl)cyclohexyl-N-ethyl oxamate, or in order for the cooling effect to be accompanied by a fresh odor, it is preferable to add at least one further cooling active ingredient.

Further products according to the invention include, in addition to 5-methyl-2-(1-methylethyl)cyclo-hexyl-N-ethyl oxamate, at least one further cooling active ingredient, which is selected from L-menthol, D-menthol and DL-menthol, preferably L-menthol, moreover from isopulegol, in particular (−)-isopulegol (FEMA 2962), N-2,3-trimethyl-2-isopropylbutamide, menthyl lactate, menthyl pyrrolidone carboxylic acid, menthyl methyl ether, menthoxypropane-1,2-diol, menthone glycerol acetal (9-methyl-6-(1-methylethyl)-1,4-dioxaspiro(4.5)-decane-2-methanol), monomenthyl succinate, 2-hydroxymethyl-3,5,5-trimethylcyclohexanol, 2-isopropyl-N,2,3-trimethylbutyramide (FEMA 3804), N-ethyl-p-menthane-3-carboxamide (FEMA 3455), in particular 1R,3R,4S-N-ethyl-p-menthane-3-carboxamide, ethyl-3-(p-menthane-3-carboxamido)acetate (FEMA 4309), (1R,2S,5R)-N-(4-methoxyphenyl)-p-menthane carboxamide (FEMA 4681), N-ethyl-2,2-diisopropylbutanamide (FEMA 4557), N-cyclopropyl-5-methyl-2-propan-2-ylcyclohexane-1-carboxamide (FEMA 4693), N-(4-cyanomethylphenyl)-p-menthane carboxamide (FEMA 4496), N-(2-(pyridin-2-yl)ethyl)-3-p-menthane carboxamide (FEMA 4549), N-(2-hydroxyethyl)-2-isopropyl-2,3-dimethylbutanamide (FEMA 4602), N-(1,1-dimethyl-2-hydroxyethyl)-2,2-diethylb utanamide (FEMA 4603), (2S,5R)-N-[4-(2-amino -2-oxo ethyl)phenyl]-p-menthane carboxamide (FEMA 4684), 2-[(2-p-menthoxy)ethoxy]ethanol (FEMA 4718), (2,6-diethyl-5-isopropyl-2-methyltetrahydropyrane (FEMA 4680), 3-(1-menthoxy)-2-methylpropane-1,2-diol (FEMA 3849), p-menthane-3,8-diol, in particular (+)-cis-p-menthane-3,8-diol and (−)-trans-p-menthane-3,8-diol as well as mixtures of (+)-cis-p-menthane-3,8-diol and (−)-trans-p-menthane-3,8-diol, in particular a mixture in the weight ratio 62:38 (FEMA 4053), (1R,3R,4S)-3-menthyl-3,6-dioxaheptanoate, (1R,2S,5R)-3-menthylmethoxyacetate, (1R,2S,5R)-3-menthyl-3,6,9-trioxadecano ate, (1R,2S,5R)-3-menthyl-3,6,9-trioxadecanoate, (1R,2S,5R)-3-menthyl-(2-hydroxyethoxy)acetate, (1R,2S,5R)-menthyl-11-hydroxy-3,6,9-trioxaundecano ate, (−)-cubebol (FEMA 4497), N-(4-cyanomethylphenyl)-p-menthane carboxamide, N,N-dimethylmenthylsuccinamide (2-isopropyl-5-methylcyclohexyl-4-(dimethylamino)-4-oxobutanoate, FEMA 4230), 6-isopropyl-3,9-dimethyl-1,4-dioxaspiro[4.5]decan-2-one (FEMA 4285), as well as mixtures of these substances, L-menthol being particularly preferred.

Products that are preferred according to the invention are characterized in that the menthol is selected from L-menthol, D-menthol and DL-menthol, preferably selected from DL-menthol. Further preferred products according to the invention include 0.09 to 5 wt. %, preferably 0.1 to 2.5 wt. %, particularly preferably 0.2 to 1.8 wt. %, exceptionally preferably 0.3 to 1.0 wt. % of menthol, selected from L-menthol, D-menthol and DL-menthol, the stated percentages by weight relating to the total weight of the water-in-oil emulsion, without taking into account any propellant that is present.

Further products that are preferred according to the invention include in total 0.05 to 3 wt. %, preferably 0.1 to 2.5 wt. %, particularly preferably 0.2 to 1.8 wt. %, exceptionally preferably 0.3 to 1.0 wt. % of one or more further cooling active ingredients, which are selected from isopulegol, in particular (−)-isopulegol (FEMA 2962), N-2,3-trimethyl-2-isopropylbutamide, menthyl lactate, menthyl pyrrolidone carboxylic acid, menthyl methyl ether, menthoxypropane-1,2-diol, menthone glycerol acetal (9-methyl-6-(1-methylethyl)-1,4-dioxaspiro(4.5)-decane-2-methanol), monomenthyl succinate, 2-hydroxymethyl-3,5,5-trimethylcyclohexanol, 2-isopropyl-N,2,3-trimethylbutyramide (FEMA 3804), N-ethyl-p-menthane-3-carboxamide (FEMA 3455), in particular 1R,3R,4S-N-ethyl-p-menthane-3-carboxamide, ethyl-3-(p-menthane-3-carboxamido)acetate (FEMA 4309), (1R,2S,5R)-N-(4-methoxyphenyl)-p-menthane carboxamide (FEMA 4681), N-ethyl-2,2-diisopropylbutanamide (FEMA 4557), N-cyclopropyl-5-methyl-2-propan-2-ylcyclohexane-1-carboxamide (FEMA 4693), N-(4-cyanomethylphenyl)-p-menthane carboxamide (FEMA 4496), N-(2-(pyridin-2-yl)ethyl)-3-p-menthane carboxamide (FEMA 4549), N-(2-hydroxyethyl)-2-isopropyl-2,3-dimethylbutanamide (FEMA 4602), N-(1,1-dimethyl-2-hydroxyethyl)-2,2-diethylbutanamide (FEMA 4603), (2S,5R)-N-[4-(2-amino-2-oxoethyl)phenyl]-p-menthane carboxamide (FEMA 4684), 2-[(2-p-menthoxy)ethoxy]ethanol (FEMA 4718), (2,6-diethyl-5-isopropyl-2-methyltetrahydropyrane (FEMA 4680), 3-(1-menthoxy)-2-methylpropane-1,2-diol (FEMA 3849), p-menthane-3,8-diol, in particular (+)-cis-p-menthane-3,8-diol and (−)-trans-p-menthane-3,8-diol as well as mixtures of (+)-cis-p-menthane-3,8-diol and (−)-trans-p-menthane-3,8-diol, in particular a mixture in the weight ratio 62:38 (FEMA 4053), (1R,3R,4S)-3-menthyl-3,6-dioxaheptanoate, (1R,2S,5R)-3-menthylmethoxyacetate, (1 R,2S,5R)-3-menthyl-3,6,9-trioxadecanoate, (1R,2S,5R)-3-menthyl-3,6,9-trioxadecano ate, (1R,2S,5R)-3-menthyl-(2-hydroxyethoxy)acetate, (1R,2 S,5R)-menthyl-11-hydroxy-3,6,9-trioxaundecanoate, (−)-cubebol (FEMA 4497), N-(4-cyanomethylphenyl)-p-menthane carboxamide, N,N-dimethylmenthylsuccinamide (2-isopropyl-5-methylcyclohexyl-4-(dimethylamino)-4-oxobutanoate, FEMA 4230), 6-isopropyl-3,9-dimethyl-1,4-dioxaspiro[4.5]decan-2-one (FEMA 4285), as well as mixtures of these substances, the stated percentages by weight relating to the total weight of the water-in-oil emulsion, without taking into account any propellant that is present.

The products according to the invention preferably include one or more preservatives. Preservatives that are preferred according to the invention are formaldehyde releasers (such as for example 1,3-dimethylol-4,4-dimethylhydantoin, INCI name DMDM Hydantoin), iodopropynyl butylcarbamates such as 3-iodo-2-propynyl butylcarbamate, parabens (i.e. para-hydroxybenzoic acid alkyl esters, such as methyl-, ethyl-, propyl- and/or butylparaben), phenoxyethanol, ethanol, benzoic acid, dibromodicyanobutane (2-bromo-2-bromomethyl glutarodinitrile), 2-bromo-2-nitro-propane-1,3-diol, imidazolidinyl urea, 5-chloro-2-methyl-4-isothiazolin-3-one, 2-chloroacetamide, benzalkonium chloride, benzyl alcohol, salicylic acid and salicylates. Particularly preferred preservatives according to the invention are selected from parabens (methyl-, ethyl-, propyl- and/or butylparaben) and/or phenoxyethanol.

The preservatives are included in the products according to the invention, relative to the total propellant-free water-in-oil emulsion, by preference in a total amount from 0.01 to 3, preferably 0.1 to 1.5 and particularly preferably 0.2 to 1.0 wt. %.

In a preferred embodiment the products according to the invention include as a deodorizing active ingredient at least one silver salt, which is preferably selected from silver sulfate, silver nitrate, silver citrate, silver dihydrogen citrate, silver lactate, silver acetate, silver malate, silver succinate, silver tartrate, silver mandelate, silver salicylate, silver gluconate, silver adipate and silver galactarate, as well as mixtures of these salts. Silver sulfate, silver citrate, silver dihydrogen citrate and silver lactate, as well as mixtures of these salts, are exceptionally preferred.

Further preferred compositions according to the invention include at least one silver salt which is preferably selected from silver sulfate, silver nitrate, silver citrate, silver dihydrogen citrate, silver lactate, silver acetate, silver malate, silver succinate, silver tartrate, silver mandelate, silver salicylate, silver gluconate, silver adipate and silver galactarate, as well as mixtures of these salts, in amounts such that silver is included in a total amount from 1 to 100 ppm, preferably 2 to 50 ppm, particularly preferably 5 to 20 ppm, exceptionally preferably 7 to 10 ppm, relative in each case to the weight of the propellant-free water-in-oil emulsion. The correspondingly necessary amount of silver salt(s) can be calculated from the molar masses of silver (107.87 g/mol) and the various silver salts—silver lactate for example has a molar mass of 196.94 g/mol.

In a further preferred embodiment the products according to the invention include as a deodorizing active ingredient at least one aromatic alcohol of the structure (AA-1),

[chemical structure]

in which
The residues R¹ to R⁶ independently of one another denote a hydrogen atom, an alkyl group having 1 to 10 carbon atoms, which can be linear or branched and can be substituted with OH groups or alkoxy groups having 1 to 5 carbon atoms, or an alkenyl group having 2 to 10 carbon atoms, which can be linear or branched and can be substituted with OH groups or alkoxy groups having 1 to 5 carbon atoms,
the residues R⁷ to R¹¹ independently of one another denote a hydrogen atom, a halogen atom, in particular a chlorine atom, or an alkyl group having 1 to 10 carbon atoms, which can be linear or branched and can be substituted with OH groups or alkoxy groups having 1 to 5 carbon atoms, in particular with a methoxy group,
m is 0 or 1, n, o, p independently of one another are integers from 0 to 10, wherein at least one of the values n, o, p≠0.

Particularly preferred products according to the invention include at least one alcohol AA-1 as described above, which is selected from anisic alcohol, 2-methyl-5-phenylpentan-1-ol, 1,1-dimethyl-3-phenylpropan-1-ol, benzyl alcohol, 2-phenylethan-1-ol, 3-phenylpropan-1-ol, 4-phenylbutan-1-ol, 5-phenylpentan-1-ol, 2-benzylheptan-1-ol, 2,2-dimethyl-3-phenylpropan-1-ol, 2,2-dimethyl-3-(3′-methylphenyl)propan-1-ol, 2-ethyl-3-phenylpropan-1-ol, 2-ethyl-3-(3′-methylphenyl)propan-1-ol, 3-(3′-chlorophenyl)-2-ethylpropan-1-ol, 3-(2′-chlorophenyl)-2-ethylpropan-1-ol, 3-(4′-chlorophenyl)-2-ethylpropan-1-ol, 3-(3′,4′-dichlorophenyl)-2-ethylpropan-1-ol, 2-ethyl-3-(2′-methylphenyl)propan-1-ol, 2-ethyl-3-(4′-methylphenyl)propan-1-ol, 3-(3′,4′-dimethylphenyl)-2-ethylpropan-1-ol, 2-ethyl-3-(4′-methoxyphenyl)propan-1-ol, 3-(3′,4′-dimethoxyphenyl)-2-ethylpropan-1-ol, 2-allyl-3-phenylpropan-1-ol and 2-n-pentyl-3-phenylpropan-1-ol as well as mixtures thereof. 2-Benzylheptan-1-ol and mixtures of 2-benzylheptan-1-ol and phenoxyethanol are exceptionally preferred. Further particularly preferred products according to the invention include at least one alcohol AA-1, as described above, in a total amount from 0.05 to 10 wt. %, preferably 0.1 to 5 wt. %, particularly preferably 0.2 to 2 wt. %, exceptionally preferably 0.3 to 1.5 wt. %, relative in each case to the weight of the water-in-oil emulsion, without taking into account the weight of the propellant. Exceptionally preferred products according to the invention include 2-benzylheptan-1-ol in a total amount from 0.05 to 1.5 wt. %, preferably 0.1 to 1 wt. %, particularly preferably 0.2 to 0.5 wt. %, relative in each case to the weight of the water-in-oil emulsion, without taking into account the weight of the propellant.

In a further preferred embodiment the products according to the invention include as a deodorizing product at least one 1,2-alkanediol having 5 to 12 C atoms, which is described by the formula HO—CH₂—CH(OHHCH₂)ₙ—CH₃, in which n denotes the numbers 2, 3, 4, 5, 6, 7, 8 or 9. 1,2-Alkanediols having 5 to 12 C atoms that are particularly preferably used according to the invention are selected from 1,2-pentanediol, 1,2-hexanediol, 1,2-octanediol, 1,2-decanediol and mixtures thereof. Mixtures of 1,2-hexandiol and 1,2-octanediol, preferably in the weight ratio 10:1 to 1:10, more preferably from 5:1 to 1:5, particularly preferably in the weight ratio 1:1, are a most particularly preferred combination according to the invention.

Preferred products according to the invention include at least one 1,2-alkanediol having 5 to 12 C atoms, which is described by the formula HO—CH₂—CH(OH)—(CH₂)ₙ—CH₃, in which n denotes the numbers 2, 3, 4, 5, 6, 7, 8 or 9, in a total amount from 0.2 to 15 wt. %, preferably 0.3 to 10 wt. %, particularly preferably 0.4 to 5 wt. % and exceptionally preferably 0.5 to 2 wt. %, relative in each case to the weight of the water-in-oil emulsion, without taking into account the weight of the propellant. Exceptionally preferred products according to the invention include 0.2 to 0.5 wt. % of 1,2-hexanediol and 0.2 to 0.5 wt. % of 1,2-octanediol, relative in each case to the weight of the water-in-oil emulsion, without taking into account the weight of the propellant.

Further preferred products according to the invention are characterized by a content of the deodorizing active ingredient 3-(2-ethylhexyloxy)-1,2-propanediol, preferably in a total amount from 0.05 to 5 wt. %, preferably 0.1 to 2 wt. %, particularly preferably 0.2 to 1.5 wt. %, exceptionally preferably 0.5 to 1.0 wt. %, relative in each case to the weight of the water-in-oil emulsion, without taking into account the weight of the propellant.

Further preferred products according to the invention are characterized by a content of tropolone (2-hydroxy-2,4,6-cycloheptatrienone) preferably in an amount from 0.001 to 0.1 wt. %, relative to the weight of the water-in-oil emulsion, without taking into account the weight of the propellant.

Further preferred products according to the invention are characterized by a content of the deodorizing active ingredient triethyl citrate. Triethyl citrate is a known deodorant active ingredient which acts as an enzyme inhibitor for esterases and lipases and thus contributes to the broad-spectrum action of products according to the invention. Preferred products according to the invention include 0.5 to 1.5 wt. %, preferably 3 to 8 wt. %, exceptionally preferably 4 to 6 wt. %, relative in each case to the weight of the water-in-oil emulsion, without taking into account the weight of the propellant.

Apocrine sweat is a complex mixture including inter alia sebum and other fats as well as steroids. Steroids themselves are not water-soluble. In order for them to be able to be transported away with the bodily fluids, they are normally present as a sulfate or glucuronide. On the skin these steroids are split into the volatile free steroids by hydrolytic enzymes of the skin bacteria, in particular the coryneform bacteria. All bacterial exoesterases are capable of this in principle, but particularly the enzymes aryl sulfatase and beta-glucuronidase. Compounds that inhibit aryl sulfatase or beta-glucuronidase are therefore preferred deodorant active ingredients according to the invention.

The development of the short- and medium-chain fatty acids which contribute substantially to body odor begins with the splitting of skin lipids into branched long-chain fatty acids. The splitting of skin lipids, which are present predominantly as glycerol esters, is carried out substantially by propionibacterium, corynebacterium A and staphylococcus species (A. G. James et al., Generation and Turnover of Volatile Fatty Acids by Axillary Bacteria, 22nd IFSCC Congress, Edinburgh, 2002, Poster 108). A. G. James et al. further disclose that both short-chain C₂-C₅ fatty acids and medium-chain C₆-C₁₂ fatty acids, which are primarily responsible for axillary body odor, are formed from the long-chain branched fatty acids by the hydrolytic enzymes of a particular corynebacterium, which A. G. James et al. call corynebacterium A. All bacterial exoesterases are capable of this lipid splitting in principle, but in particular the enzyme lipase. Therefore, compounds which inhibit lipases are likewise preferred deodorant active ingredients according to the invention.

A further class of compounds which is likewise formed during bacterial degradation of the sweat components and contributes to body odor are saturated and unsaturated aldehydes, above all those having a chain length of C₆ to C₁₂, in particular hexanal, heptanal, octenal and nonenal. These are formed by beta-cleavage from the hydroperoxides which are formed under the influence of 5-lipoxigenase on unsaturated fatty acids. Therefore, compounds which inhibit the enzyme 5-lipoxigenase are likewise preferred deodorant active ingredients according to the invention.

It is moreover known that volatile sulfur compounds (VSCs), which are released in particular by enzymatic reaction, are highly malodorous components of human body odor and halitosis. Sulfur-containing compounds occur as water-soluble amino acid conjugates with the sweat on human skin. There they are released by skin bacteria (above all by staphylococci and corynebacteria) by enzymatic reaction. An enzyme that plays a particular part in the release of VSCs is cystathionine beta-lyase. This enzyme splits off VSCs from the amino acids and is thus an important cause in the formation of body odor. Therefore, compounds which inhibit the enzyme cystathionine beta-lyase are likewise preferred deodorant active ingredients according to the invention.

Further preferred products according to the invention are characterized by a content of at least one compound that is an inhibitor of the enzyme aryl sulfatase. Preferred deodorant active ingredients which act as an aryl sulfatase inhibitor are those disclosed in U.S. Pat. No. 5,643,559, U.S. Pat. No. 5,676,937, WO2001/099376 A2, EP 1430879 A1 and DE 10216368 A1. Further particularly preferred products according to the invention are characterized by a content of at least one compound which is an inhibitor of the enzyme aryl sulfatase, in a total amount from 0.001 to 10 wt. %, preferably 0.01 to 5 wt. %, particularly preferably 0.1 to 2.5 wt. %, relative in each case to the weight of the water-in-oil emulsion, without taking into account the weight of the propellant.

Further preferred products according to the invention are characterized by a content of at least one compound that is an inhibitor of the enzyme beta-glucuronidase. Preferred deodorant active ingredients according to the invention which act as a beta-glucuronidase inhibitor are those disclosed in WO2003/039505 A2. Further particularly preferred products according to the invention are characterized by a content of at least one compound which is an inhibitor of the enzyme beta-glucuronidase, in a total amount from 0.001 to 10 wt. %, preferably 0.01 to 5 wt. %, particularly preferably 0.1 to 2.5 wt. %, relative in each case to the weight of the water-in-oil emulsion, without taking into account the weight of the propellant.

Further preferred products according to the invention are characterized by a content of at least one compound that is an inhibitor of the enzyme lipase. Preferred deodorant active ingredients which act as a lipase inhibitor are selected from those disclosed in EP 1428520 A2, selected moreover from the aminomethylene malonic acid derivatives according to DE 3018132 A1, the ethylene oxide-propylene oxide copolymers according to GB 2335596 A1 and the salts of phytic acid according to EP 650 720 A1. Further particularly preferred products according to the invention are characterized by a content of at least one compound which is an inhibitor of the enzyme lipase, in a total amount from 0.001 to 10 wt. %, preferably 0.01 to 5 wt. %, particularly preferably 0.1 to 2.5 wt. %, relative in each case to the weight of the water-in-oil emulsion, without taking into account the weight of the propellant.

Further particularly preferred products according to the invention are characterized by a content of at least one compound that is an inhibitor of 5-lipoxigenase. Preferred deodorant active ingredients according to the invention which act as a 5-lipoxigenase inhibitor are disclosed in EP 1428519 A2. Further particularly preferred products according to the invention are characterized by a content of at least one compound which is an inhibitor of the enzyme 5-lipoxigenase, in a total amount from 0.001 to 10 wt. %, preferably 0.01 to 5 wt. %, particularly preferably 0.1 to 2.5 wt. %, relative in each case to the weight of the water-in-oil emulsion, without taking into account the weight of the propellant.

Further particularly preferred products according to the invention are characterized by a content of at least one compound that is an inhibitor of the enzyme cystathionine beta-lyase. Preferred deodorant active ingredients according to the invention which act as an inhibitor of cystathionine beta-lyase are selected from those disclosed in EP 495918 B1, WO 2006/079934, DE 102010000746 A1, WO2010/031657 A1 and WO2010/046291 A1. Further particularly preferred products according to the invention are characterized by a content of at least one compound which is an inhibitor of the enzyme cystathionine beta-lyase, in a total amount from 0.001 to 10 wt. %, preferably 0.01 to 5 wt. %, particularly preferably 0.1 to 2.5 wt. %, relative in each case to the weight of the water-in-oil emulsion, without taking into account the weight of the propellant.

A further optional constituent of products according to the invention is at least one cationic phospholipid of formula KPL,

[chemical structure]

in which R¹ is an alkyl, alkenyl or hydroxyalkyl group having 8 to 22 C atoms or an acyl aminoalkyl group of the formula R⁵CONH(CₘH₂ₘ)—, in which R⁵CO is a linear acyl group having 8 to 22 C atoms and m=2 or 3, R² and R³ are alkyl groups having 1 to 4 C atoms or hydroxyalkyl groups having 2 to 4 C atoms or carboxyalkyl groups of the formula —(CH₂)_{z}—COOM, in which z is a value from 1 to 3 and M is hydrogen or an alkali metal cation, x has a value from 1 to 3 and y has a value of (3−x), M is hydrogen or an alkali metal cation and A⁻ is an anion.

Preferred alkyl groups having 8 to 22 C atoms are selected from an n-octyl, n-nonyl, n-decyl, n-undecyl, lauryl, n-tridecanyl, myristyl, n-pentadecanyl, cetyl, palmityl, stearyl, elaidyl, arachidyl, behenyl and a cocyl group. A representative cocyl group consists, relative to its total weight, of 4 to 9 wt. % of n-octyl, 4 to 9 wt. % of n-decyl, 45 to 55 wt. % of lauryl, 15 to 21 wt. % of myristyl, 8 to 13 wt. % of palmityl and 7 to 14 wt. % of stearyl groups. Preferred alkenyl groups having 8 to 22 C atoms are selected from a linoleyl group ((9Z,12Z)-octadeca-9,12-dien-1-yl) and a linolenyl group ((9Z,12Z,15Z)-octadeca-9,12,15-trien-1-yl). A preferred hydroxyalkyl group having 8 to 22 C atoms is selected from a 12-hydroxystearyl group.

Particularly preferred cationic phospholipids of formula KPL are those in which R¹ denotes an acyl aminoalkyl group of the formula R⁵CONH(CₘH₂ₘ)—, in which R⁵CO is a linear acyl group having 8 to 22 C atoms and m=3.

Preferred linear acyl groups R⁵CO having 8 to 22 C atoms are selected from an n-octanoyl, n-nonanoyl, n-decanoyl, n-undecanoyl, lauroyl, n-tridecanoyl, myristoyl, n-pentadecanoyl, cetoyl, palmitoyl, stearoyl, elaidoyl, arachidoyl, behenoyl and a cocoyl group. A representative cocoyl group consists, relative to its total weight, of 4 to 9 wt. % of n-octanoyl, 4 to 9 wt. % of n-decanoyl, 45 to 55 wt. % of lauroyl, 15 to 21 wt. % of myristoyl, 8 to 13 wt. % of palmitoyl and 7 to 14 wt. % of stearoyl groups. Particularly preferred linear acyl groups R⁵CO are selected from a cocoyl group, a lauroyl group (n-C₁₁H₂₃CO), a myristoyl group (n-C₁₃H₂₇CO) and a linoleoyl group ((9Z,12Z)-octa-deca-9,12-dien-1-oyl). Exceptionally preferred linear acyl groups R⁵CO are selected from a cocoyl group, a lauroyl group (n-C₁₁H₂₃CO) and a myristoyl group (n-C₁₃H₂₇CO).

Preferred alkyl groups having 1 to 4 C atoms are a methyl, ethyl, n-propyl, 1-methylethyl, n-butyl, 2-methylpropyl and tert-butyl group. The methyl group is particularly preferred.

Preferred hydroxyalkyl groups having 2 to 4 C atoms are a 2-hydroxyethyl and a 1-hydroxyethyl group.

Preferred carboxyalkyl groups of the formula —(CH₂)_{z}—COOM with z=1 to 3 are a carboxymethyl, a carboxyethyl and a carboxy-n-propyl group.

Preferred alkali metal cations are selected from sodium and potassium cations; Na⁺ is particularly preferred. Preferred anions are selected from sulfate, chloride, phosphate, nitrate, hydrogen carbonate and acetate, a chloride anion being particularly preferred.

Preferred products according to the invention include as a deodorizing active ingredient a cationic phospholipid of formula KPL

[chemical structure]

in which R¹ is an acyl aminoalkyl group of the formula R⁵CONH(CₘH₂ₘ)—, in which R⁵CO is selected from a cocoyl group, a lauroyl group, a myristoyl group and a linoleoyl group and m=3, R² and R³ are methyl groups, x=2, y=1, M is a sodium ion and A⁻ is a chloride ion. At least one cationic phospholipid of formula KPL with the aforementioned features is preferably included in a total amount from 0.05 to 2 wt. %, preferably 0.1 to 1 wt. %, particularly preferably 0.15 to 0.4 wt. %, relative in each case to the weight of the water-in-oil emulsion, without taking into account any propellant that is included.

Particularly preferred products according to the invention include a cationic phospholipid of formula KPL

[chemical structure]

in which R¹ is a cocoyl aminopropyl group (also referred to as a cocamidopropyl group), R² and R³ are methyl groups, x=2, y=1, M is a sodium ion and A⁻ is a chloride ion and which is obtainable under the INCI name Cocamidopropyl PG-Dimonium Chloride Phosphate, in a total amount from 0.05 to 2 wt. %, preferably 0.1 to 1 wt. %, particularly preferably 0.15 to 0.4 wt. %, relative in each case to the weight of the water-in-oil emulsion, without taking into account any propellant that is included.

Further particularly preferred products according to the invention include a cationic phospholipid of formula KPL

[chemical structure]

in which R¹ is a myristoyl aminopropyl group, R² and R³ are methyl groups, x=2, y=1, M is a sodium ion and A⁻ is a chloride ion and which is obtainable under the INCI name Myristamidopropyl PG-Dimonium Chloride Phosphate, in a total amount from 0.05 to 2 wt. %, preferably 0.1 to 1 wt. %, particularly preferably 0.15 to 0.4 wt. %, relative in each case to the weight of the water-in-oil emulsion, without taking into account any propellant that is included.

Further particularly preferred products according to the invention include a cationic phospholipid of formula KPL

[chemical structure]

in which R¹ is a lauroyl aminopropyl group, R² and R³ are methyl groups, x=2, y=1, M is a sodium ion and A⁻ is a chloride ion, in a total amount from 0.05 to 2 wt. %, preferably 0.1 to 1 wt. %, particularly preferably 0.15 to 0.4 wt. %, relative in each case to the weight of the water-in-oil emulsion, without taking into account any propellant that is included.

The water-in-oil emulsion according to the invention is contained together with at least one propellant in an aerosol dispensing device; the cosmetic product according to the invention consists of the water-in-oil emulsion as described above, at least one propellant and an aerosol dispensing device.

Propellants (propellant gases) which are suitable according to the invention are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, both individually and in combination. Hydrophilic propellant gases such as carbon dioxide for example can also advantageously be used within the meaning of the present invention if the chosen proportion of hydrophilic gases is small and lipophilic propellant gas (e.g. propane/butane) is present in excess. Propane, n-butane, isobutene and mixtures of these propellant gases are particularly preferred.

Further propellants which are suitable and preferred according to the invention are selected from at least one compound having 3 to 10 carbon atoms according to formula (I)

[chemical structure]

in which the residues R¹, R², R³ and R⁴ independently of one another denote a hydrogen atom, a bromine atom, a fluorine atom or a (C₁ to C₆) alkyl group substituted with at least one fluorine atom, or two of the residues R¹, R², R³ and R⁴ form a five- or six-membered ring, with the proviso that at least one of the residues R¹, R², R³ or R⁴ denotes a hydrogen atom or a fluorine atom and at least one of the residues R¹, R², R³ or R⁴ denotes a (C₁ to C₆) alkyl group substituted with at least one fluorine atom or at least two of the residues R¹, R², R³ and R⁴ form a five- or six-membered ring.

Preferred propellants are selected from at least one compound having 3 to 10 carbon atoms according to formula (I) above, in which R¹, R², R³ or R⁴ independently of one another denote a hydrogen atom, a fluorine atom or a (C₁ to C₆) alkyl group substituted with at least one fluorine atom, with the proviso that at least one of the residues R¹, R², R³ or R⁴ denotes a hydrogen atom or a fluorine atom and at least one of the residues R¹, R², R³ or R⁴ denotes a (C₁ to C₆) alkyl group substituted with at least one fluorine atom. Particularly preferred propellants are selected from compounds of the formula E-R¹CH═CHR² or of the formula Z-R¹CH═CHR², in which R¹ and R² independently of one another denote a perfluorinated C₁ to C₆ alkyl group.

Further particularly preferred propellants are selected from CF₃CF═CHF, CF₃CH═CF₂, CHF₂CF═CF₂, CHF₂CH═CHF, CF₃CF═CH₂, CF₃CH═CHF, CH₂FCF═CF₂, CHF₂CH═CF₂, CHF₂CF═CHF, CHF₂CF═CH₂, CF₃CH═CH₂, CH₃CF═CF₂, CH₂FCH═CF₂, CH₂FCF═CHF, CHF₂CH═CHF, CF₃CF═CFCF₃, CF₃CF₂CF═CF₂, CF₃CF═CHCF₃, CF₃CF₂CF═CH₂, CF₃CH═CHF₃, CF₃CF₂CH═CH₂, CF₂═CHCF₂CF₃, CF₂═CHCF₂CF₃, CF₂═CFCHFCF₃, CF₂═CFCF₂CHF₂, CHF₂CH═CHCF₃, (CF₃)₂C═CHCF₃, CF₃ CF═CHCF₂CF₃, CF₃ CH═CFCF₂CF₃, (CF₃)₂CFCH═CH₂, CF₃CF₂CF₂CH═CH₂, CF₃(CF₂)₃CF═CF₂, CF₃ CF₂CF═CFCF₂CF₃, (CF₃)₂C═C(CF₃)₂, (CF₃)₂CFCF═CHCF₃, CF₂═CFCF₂CH₂F, CF₂═CFCHFCHF₂, CH₂═C(CF₃)₂, CH₂CF₂CF═CF₂, CH₂FCF═CFCHF₂) CH₂FCF₂CF═CF₂, CF₂═C(CF₃)(CH₃), CH₂═C(CHF₂)(CF₃), CH₂═CHCF₂CHF₂, CF₂═C(CHF2)(CH3), CHF═C(CF₃)(CH₃), CH₂—C(CHF₂)₂, CF₃CF═CFCH₃, CH₃CF═CHCF₃, CF₂═CF(CF₂)₂CF₃, CHF═CF(CF₂)₂CF₃, CF₂═CH(CF₂)₂CF₃, CF₂═CF(CF₂)₂CHF₂, CHF₂CF═CFCF₂CF₃, CF₃CF═CFCF₂CHF₂, CF₃CF═CFCHFCF₃, CHF═CFCF(CF₃)₂, CF₂═CFCH(CF₃)₂, CF₃CH═C(CF₃)₂, CF₂═CHCF(CF₃)₂, CH₂═CF(CF₂)₂CF₃) CHF═CF(CF₂)₂CHF₂, CH₂═C(CF₃)C₂F₅, CF₂═CHCH(CF₃)₂, CHF═CHCF(CF₃)₂, CF₂═C(CF₃)CH₂CF₃, CH₂═CF(CF₂)₂CHF₂, CF₂—CHCF₂CH₂CF₃, CF₃CF═C(CF₃)CH₃, CH₂═CFCH(CF₃)₂, CHF═CHCH(CF₃)₂, CH₂FCH═C(CF₃)₂, CH₃CF═C(CF₃)₂, CH₂═CHCF₂CHFCF₃, CH₂═C(CF₃)CH₂CF₃, (CF₃)₂C═CHC₂F₅, CH₂═CHC(CF₃)₃, (CF₃)₂C═C(CH₃)CF₃, CH₂═CFCF₂CH(CF₃)₂, CF₃CF═C(CH₃)C₂F₅, CF₃CH═CHCH(CF₃)₂, CH₂═CH(CF₂)₃CHF₂, (CF₃)₂C═CHCF₂CH₃, CH₂—C(CF₃)CH₂C₂F₅, CH₂═CHCH₂CF₂CF₂CF₃, C₂F₅CF═CFC₂H₅, CH₂═CHCH₂CF(CF₃)₂, CF₃CF═CHCH(CF₃)(CH₃), (CF₃)₂C═CFC₂H₅, cyclo-CF₂CF₂CF₂CH═CH—, cyclo-CF₂CF₂CH═CH—, CF₃CF₂CF₂C(CH₃)—CH₂, CF₃CF₂CF₂CH═CHCH₃, cyclo-CF₂CF₂CF═CF—, cyclo-CF₂CF═CFCF₂CF₂—, cyclo-CF₂CF═CFCF₂CF₂CF₂—, CF₃CF₂CF₂CF₂CH═CH₂, CF₃CH═CHC₂F₅, C₂F₅CH═CHC₂F₅, CF₃CH═CHCF₂CF₂CF₃, CF₃CF═CFC₂F₅, CF₃CF═CFCF₂CF₂CF₂CF₃, C₂F₅CF═CFCF₂CF₂CF₃, CF₃CH═CFCF₂CF₂CF₂CF₃, CF₃CF═CHCF₂CF₂CF₂CF₃, C₂F₅CH═CFCH₂CH₂CH₃, C₂F₅CF═CHCF₂CF₂CF₃, CF₃CF₂CF₂CF═CHCH₃, C₂F₅CF═CHCH₃, (CF₃)₂OCHCH₃, CF₃C(CH₃)═CHCF₃, CHF═CFC₂F₅, CHF₂CF═CFCF₃, (CF₃)₂C═CHF, CH₂FCF═CFCF₃, CHF═CHC₂F₅, CHF₂CH═CFCF₃, CHF═CFCHFCF₃, CF₃CH═CFCHF₂, CHF═CFCF₂CHF₂, CHF₂CF═CFCHF₂, CH₂CF═CFCF₃, CH₂FCH═CFCF₃, CH₂═CFCHFCF₃, CH₂═CFCF₂CHF₂, CF₃CH═CFCH₂F, CHF═CFCH₂CF₃, CHF═CHCHFCF₃, CHF═CHCF₂CHF₂, CHF₂CF═CHCHF₂, CHF═CFCHFCHF₂, CF₃CF═CHCH₃, CF₂═CHCF₂Br, CHF═CBrCHF₂, CHBr═CHCF₃, CF₃CBr—CFCF₃, CH₂═CBrC₂F₅, CHBr═CHC₂F₆, CH₂═CH(CF₂)₂Br, CH₂═CHCBrFCF₃, CH₃CBr—CHCF₃, CF₃CBr═CHCH₃, (CF₃)₂C═CHBr, CF₃CF═CBrC₂F₅, E-CHF₂CBr═CFC₂F₅, Z-CHF₂CBr═CFC₂F₅, CF₂═CBrCHFC₂F₅, (CF₃)₂CFCBr═CH₂, CHBr═CF(CF₂)₂CHF₂, CH₂═CBrCF₂CF₂CF₃, CF₂═C(CH₂Br)CF₃, CH₂═C(CBrF₂)CF₃, (CF₃),CHCH═CHBr, (CF₃)₂C═CHCH₂Br, CH₂═CHCF(CF₃)CBrF₂, CF₂═CHCF₂CH₂CBrF₂, CFBr═CHCF₃, CFBr═CFCF₃ and CH₂═CBrCF₂CF₂CF₂CF₃, in each case in the E form or the Z form, as well as mixtures of the aforementioned components.

Particularly preferred cosmetic products according to the invention include E-CF₃CH═CHF (E-1,3,3,3-tetrafluoroprop-1-ene) as the propellant of formula (I).

Cosmetic products which include the propellant in an amount from 10 to 90 wt. %, preferably 40 to 85 wt. % and particularly preferably 50 to 80 wt. %, relative in each case to the total weight of the preparation consisting of the water-in-oil emulsion and the propellant are preferred according to the invention.

Vessels made from metal (aluminum, white metal, tin), protected or shatterproof plastic or from glass externally coated with plastic are suitable as pressurized-gas containers, in the selection of which compressive strength and fracture strength, corrosion resistance, ease of filling and also aesthetic factors, manageability, printability, etc., play a part. Special interior protective lacquers ensure resistance to corrosion from the water-in-oil emulsion.

Despite the water phase in the aerosol container, the products according to the invention have a particularly high corrosion resistance, and this represents a great advantage over the prior art. Furthermore, the water-in-oil emulsions used according to the invention have excellent skin compatibility, storage stability and effectiveness. It is advantageous in particular that the sprayed products are characterized on the skin by a pleasant, non-sticky skin feel with a long-lasting cooling effect.

In a preferred embodiment of the invention the valve has a valve disk coated with a lacquer or a polymeric plastic A and a similarly flexible element having resetting characteristics, which on completion of actuation resets the valve to the closed position (=rest position of the valve). Corresponding cosmetic products in which the aerosol dispensing device comprises a valve having a valve plug and/or a flexible element with resetting characteristics, which is/are coated with a lacquer or a polymeric plastic A are preferred according to the invention.

In a further preferred embodiment of the invention the valve has a flexible element with resetting characteristics and/or a valve disk made from at least one plastic B, preferably an elastomeric plastic. In this case too cosmetic products according to the invention are preferred in which the valve has a flexible element with resetting characteristics and/or a valve plug made from at least one plastic B, preferred plastics B being elastomeric plastics. Particularly preferred elastomeric plastics are selected from Buna, in particular Buna N, Buna 421, Buna 1602 and Buna KA 6712, Neoprene, butyl and chlorobutyl.

In a further preferred embodiment of the invention the flexible element with resetting characteristics can be designed as a spiral spring or helical compression spring. In a further preferred embodiment of the invention the flexible element with resetting characteristics can be designed as being integral with the valve plug and can have flexible legs. This spring can be made from metal, preferably from highly hardened and tempered, corrosion-resistant stainless steel (e.g. grade AISI 316) or plastic.

In a particularly preferred embodiment of the invention the valve disk and flexible element are designed with resetting characteristics. The valve type Ariane M, available from Seaquist Perfect, is particularly preferred, in which the flexible element with resetting characteristics is designed in the form of four elastic legs which are integral with the valve plug. All valves that are used according to the invention preferably have a valve disk that is lacquered on the inside, the lacquer and valve material being mutually compatible. If aluminum valves are used according to the invention, the valve disks can be coated on the inside with Micoflex lacquer, for example. If white metal valves are used according to the invention, the valve disks can be coated on the inside with PET (polyethylene terephthalate), for example. The containers that are used, which can be made for example from white metal or aluminum, aluminum containers being preferred according to the invention, must likewise be lacquered or coated on the inside in view of the corrosivity of the water-in-oil emulsions used according to the invention. An interior protective lacquer that is preferred according to the invention is an epoxy-phenolic lacquer such as is obtainable inter alia under the names Hoba 7407 P and Hoba 7940.

The cosmetic products according to the invention are suitable for treating the skin. The present application therefore also provides a method for the cosmetic treatment of the skin, characterized in that a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20, preferably in the range from >12 to 19, particularly preferably in the range from >14 to 18 and exceptionally preferably in the range from 15 to 17,
d) at least one cosmetic oil that is not a fragrance and not an essential oil,
e) 5-methyl-2-(1-methylethyl)cyclohexyl-N-ethyl oxamate, preferably (1R,2S,5R)-5-methyl-2-(1-methylethyl)cyclohexyl-N-ethyl oxamate,
   is applied to the skin with the help of a propellant from an aerosol dispensing device. All that has been stated in respect of the products and water-in-oil emulsions according to the invention applies with necessary alterations to further preferred embodiments of the method according to the invention.

The examples below are intended to clarify the subject matter of the invention without restricting its scope thereto.

Water-in-oil emulsions according to the invention (all stated amounts in wt. %, relative to the total weight of the water-in-oil emulsion without propellant)

| | 1 | 2 | 3 | 4 |
|---|---|---|---|---|
| Aluminum chlorohydrate | 33 | 33 | 25 | 25 |
| C₁₀-C₁₃ Isoalkane | 8.9 | 8.9 | 8.9 | 8.9 |
| PEG/PPG-18/18 Dimethicone* | 1.4 | 1.4 | 1.4 | 1.4 |
| lsoceteth-20 | 0.50 | 0.50 | 0.50 | 0.50 |
| Dimethicone* | 4.2 | 4.2 | 4.2 | 4.2 |
| Isopropyl myristate | 9.0 | 9.0 | 9.0 | 9.0 |
| 1,2-Propanediol | 7.0 | 25 | 7.0 | 25 |
| Phenoxyethanol | 0.50 | 0.50 | 0.50 | 0.50 |
| Perfume | 2.5 | 2.5 | 2.5 | 2.5 |
| 5-Methyl-2-(1-methylethyl)cyclo- | | 1.2 | | |
| hexyl-N-ethyl oxamate | | | | |
| (1R,2S,5R)-5-Methyl-2-(1-methyl- | 0.1 | | 0.5 | 1 |
| ethyl)cyclohexyl-N-ethyl oxamate | | | | |
| Water | to 100 | to 100 | to 100 | to 100 |
| | 5 | 6 | 7 | 8 |
| Aluminum chlorohydrate | 25 | 25 | 25 | 25 |
| C₁₀-C₁₃ Isoalkane | 7 | 7 | 7 | 9 |
| PEG/PPG-18/18 Dimethicone* | 1 | 1 | 1 | 1.6 |
| lsoceteth-20 | 0.30 | 0.30 | 0.30 | 0.9 |
| Dimethicone* | 3 | 3 | 3 | 3 |
| 2-Ethylhexyl palmitate | 9.0 | — | — | |
| Isopropyl myristate | — | 9.0 | 6.2 | 9 |
| Di-(2-ethylhexyl)carbonate | — | — | 2.8 | |
| 1,2-Propanediol | 30 | 22 | 22 | 25 |
| Phenoxyethanol | 0.50 | 0.50 | 0.50 | 0.5 |
| Perfume | 2.5 | 2.5 | 2.5 | 2.5 |
| Silver lactate | — | — | 0.0009 | |
| Menthyl lactate | — | 1.0 | — | |
| DL-Menthol | 0.2 | — | — | 0.7 |
| (1R,2S,5R)-5-Methyl-2-(1-methyl- | 0.5 | 0.1 | 0.7 | 0.9 |
| ethyl)cyclohexyl-N-ethyl oxamate | | | | |
| Water | to 100 | to 100 | to 100 | to 100 |

*from Dow Corning ES-5227 DM

The water-in-oil emulsions according to the invention, numbers 1 to 8, are introduced into an aluminum spray can coated with an epoxy phenolic lacquer in a weight ratio of propellant (butane/propane/isobutane mixture) to emulsion of 80:20 or 85:15 or 60:40 to form a product according to the invention. After being sprayed onto the skin of the armpit they leave behind a long-lasting cooling effect.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic product consisting of
a water-in-oil emulsion including
a) at least one sweat-inhibiting aluminum salt,
b) at least one water-in-oil emulsifier having an HLB value in the range from 1.5 to 6.5,
c) at least one oil-in-water emulsifier or solubilizer having an HLB value in the range from >7 to 20,
d) at least one cosmetic oil that is not a fragrance and not an essential oil, and
e) 5-methyl-2-(1-methylethyl)cyclohexyl-N-ethyl oxamate,
at least one propellant, and
an aerosol dispensing device.

2. The product according to claim 1, wherein the 5-methyl-2-(1-methylethyl)cyclohexyl-N-ethyl oxamate, is included in a total amount from 0.05 to 2 wt. % wherein all stated amounts relate to the weight of the water-in-oil emulsion, without taking into account the weight of the propellant.

3. The product according to claim 1, wherein the water-in-oil emulsifier b) is selected from the group consisting of poly-(C₂-C₃)alkylene glycol-modified silicones, on condition that such emulsifiers have an HLB value under normal conditions in the range from 1.5 to 6.5.

4. The product according to claim 1, wherein the at least one water-in-oil emulsifier b) is included in a total amount from 0.2 to 4.0 wt. %, relative in each case to the weight of the total propellant-free water-in-oil emulsion.

5. The product according to claim 1, wherein the at least one oil-in-water emulsifier or solubilizer c) having an HLB value in the range from >7 to 20 is included in a total amount from 0.1 to 2.0 wt. % relative to the weight of the total propellant-free water-in-oil emulsion.

6. The product according to claim 1, wherein the oil-in-water emulsifier or solubilizer having an HLB value in the range from >7 to 20 is selected from non-ionic polyethylene glycol ethers.

7. The product according to claim 6, wherein the at least one non-ionic polyethylene glycol ether is selected from the group consisting of ethoxylated C₈-C₂₄ alkanols having on average 10 to 100 mol of ethylene oxide per mol.

8. The product according to claim 1, wherein the at least one oil-in-water emulsifier or solubilizer having an HLB value in the range from >7 to 20 is selected from the group consisting of Isoceteth-12, Isoceteth-20, Isoceteth-30, Isosteareth-12, Isosteareth-20, Isosteareth-30, Laureth-12 and Beheneth-20.

9. The product according to claim 1, wherein the at least one oil-in-water emulsifier or solubilizer having an HLB value in the range from >7 to 20 is Isoceteth-20.

10. The product according to claim 9, wherein the Isoceteth-20 is included in a total amount from 0.1 to 2.0 wt. % relative to the total propellant-free water-in-oil emulsion.

11. The product according to claim 1, wherein the at least one cosmetic oil d) is included in a total amount from 1 to 30 wt. % relative to the weight of the total propellant-free water-in-oil emulsion.

12. The product according to claim 1, wherein the cosmetic oil d) comprises at least one volatile oil having a vapor pressure from 10 to 3000 Pa at 20° C. in a total amount from 10 to 100 wt. % relative to the total weight of the cosmetic oils d).

13. The product according to claim 1, wherein the volatile oil d) having a vapor pressure from 10 to 3000 Pa at 20° C. is selected from the group consisting of C₈-C₁₆ isoparaffins.

14. The product according to claim 1, further comprising at least one cooling active ingredient selected from the group consisting of L-menthol, D-menthol, DL-menthol, isopulegol, N-2,3-trimethyl-2-isopropylbutamide, menthyl lactate, menthyl pyrrolidone carboxylic acid, menthyl methyl ether, menthoxypropane-1,2-diol, menthone glycerol acetal (9-methyl-6-(1-methylethyl)-1,4-dioxaspiro(4.5)-decane-2-methanol), monomenthyl succinate, 2-hydroxymethyl-3,5,5-trimethylcyclohexanol, 2-isopropyl-N,2,3-trimethylbutyramide (FEMA 3804), N-ethyl-p-menthane-3-carboxamide (FEMA 3455), ethyl-3-(p-menthane-3-carboxamido)acetate (FEMA 4309), (1R,2S,5R)-N-(4-methoxyphenyl)-p-menthane carboxamide (FEMA 4681), N-ethyl-2,2-diisopropylbutanamide (FEMA 4557), N-cyclopropyl-5-methyl-2-propan-2-ylcyclohexane-1-carboxamide (FEMA 4693), N-(4-cyanomethylphenyl)-p-menthane carboxamide (FEMA 4496), N-(2-(pyridin-2-yl)ethyl)-3-p-menthane carboxamide (FEMA 4549), N-(2-hydroxyethyl)-2-isopropyl-2,3-dimethylbutanamide (FEMA 4602), N-(1,1-dimethyl-2-hydroxyethyl)-2,2-diethylbutanamide (FEMA 4603), (2S,5R)-N-[4-(2-amino-2-oxoethyl)phenyl]-p-menthane carboxamide (FEMA 4684), 2-[(2-p-menthoxy)ethoxy]ethanol (FEMA 4718), (2,6-diethyl-5-isopropyl-2-methyltetrahydropyrane (FEMA 4680), 3-(1-menthoxy)-2-methylpropane-1,2-diol (FEMA 3849), p-menthane-3,8-diol, (1R,3R,4S)-3-menthyl-3,6-dioxaheptanoate, (1R,2S,5R)-3-menthylmethoxyacetate, (1R,2S,5R)-3-menthyl-3,6,9-trioxadecanoate, (1R,2 S,5R)-3-menthyl-3,6,9-trioxadecano ate, (1R,2S,5R)-3-menthyl-(2-hydroxyethoxy)acetate, (1R,2S,5R)-menthyl-11-hydroxy-3,6,9-trioxaundecanoate, (−)-cubebol (FEMA 4497), N-(4-cyanomethylphenyl)-p-menthane carboxamide, N,N-dimethylmenthylsuccinamide (2-isopropyl-5-methylcyclohexyl-4-(dimethylamino)-4-oxobutanoate, FEMA 4230), 6-isopropyl-3,9-dimethyl-1,4-dioxaspiro [4.5]decan-2-one (FEMA 4285), and mixtures thereof.

15. The product according to claim 1, wherein the at least one sweat-inhibiting aluminum salt is included in a total amount from 5 to 40 wt. % relative to the total weight of the active substance (USP), free from water of crystallization and ligands, in the propellant-free water-in-oil emulsion.
